(19) United States
(12) Patent Application Publication
(10) Pub. No.: US 2022/0130516 A1
(43) Pub. Date: Apr. 28, 2022

(54) METHOD AND SYSTEM FOR DYNAMICALLY GENERATING CULTURALLY SENSITIVE PROFILE-SPECIFIC THERAPEUTIC PROTOCOLS USING MACHINE LEARNING MODELS

(71) Applicant: Mahana Therapeutics, Inc., San Francisco, CA (US)

(72) Inventor: Simon Levy, Denver, CO (US)

(73) Assignee: Mahana Therapeutics, Inc., San Francisco, CA (US)

(21) Appl. No.: 17/503,037

(22) Filed: Oct. 15, 2021

Related U.S. Application Data
(60) Provisional application No. 63/104,322, filed on Oct. 22, 2020.

(51) Int. Cl. G16H 20/70; G16H 40/67; G16H 50/70; G06N 3/08; G16H 10/20

(52) U.S. Cl. CPC G16H 20/70; G16H 40/67; G16H 10/20; G06N 3/08; G16H 50/70

(57) ABSTRACT

A protocol generation system for a first culture takes historical therapeutic protocol data associated with a first culture as input data, and outputs maximally effective therapeutic protocols for patients associated with the first culture. The maximally effective therapeutic protocols for patients associated with the first culture are provided to a protocol translation module, resulting in the generation of translated therapeutic protocols for cultures other than the first culture. The translated therapeutic protocols are provided to a culturally sensitive protocol translation module, which transforms the translated therapeutic protocols into protocols that are culturally sensitive to cultures other than the first culture. Individual protocol generation systems associated with cultures other than the first culture are utilized to generate maximally effective therapeutic protocols for patients associated with cultures other than the first culture.

### RELATED APPLICATIONS

This application claims the benefit of Paull et al., U.S. Provisional Patent Application No. 63/104,322, filed on Oct. 22, 2020, entitled “SYSTEMS AND METHODS FOR GUIDED ADMINISTRATION OF BEHAVIORAL THERAPY,” which is hereby incorporated by reference in its entirety as if it were fully set forth herein.

This application is related to U.S. patent application Ser. No. 17/216,993 (attorney docket number MAH008), naming Simon Levy as inventor, filed Mar. 30, 2021, entitled “METHOD AND SYSTEM FOR DYNAMICALLY GENERATING PROFILE-SPECIFIC THERAPEUTIC PROTOCOLS USING MACHINE LEARNING MODELS,” which is hereby incorporated by reference in its entirety as if it were fully set forth herein. This application is also related to U.S. patent application Ser. No. 17/217,001 (attorney docket number MAH009), naming Simon Levy as inventor, filed on Mar. 30, 2021, entitled “METHOD AND SYSTEM FOR DYNAMICALLY GENERATING GENERALIZED THERAPEUTIC PROTOCOLS USING MACHINE LEARNING MODELS,” which is hereby incorporated by reference in its entirety as if it were fully set forth herein. This application is also related to U.S. patent application Ser. No. 17/217,052 (attorney docket number MAH010), naming Simon Levy as inventor, filed on Mar. 30, 2021, entitled “METHOD AND SYSTEM FOR DYNAMICALLY GENERATING PROFILE-SPECIFIC THERAPEUTIC IMAGERY USING MACHINE LEARNING MODELS,” which is hereby incorporated by reference in its entirety as if it were fully set forth herein. This application is also related to U.S. patent application Ser. No. 17/217,061 (attorney docket number MAH011), naming Simon Levy as inventor, filed on Mar. 30, 2021, entitled “METHOD AND SYSTEM FOR DYNAMICALLY GENERATING GENERALIZED THERAPEUTIC IMAGERY USING MACHINE LEARNING MODELS,” which is hereby incorporated by reference in its entirety as if it were fully set forth herein. This application is also related to U.S. patent application Ser. No. ______ (attorney docket number MAH013), naming Simon Levy as inventor, filed concurrently with the present application on Oct. 15, 2021, entitled “METHOD AND SYSTEM FOR DYNAMICALLY GENERATING CULTURALLY SENSITIVE GENERALIZED THERAPEUTIC PROTOCOLS USING MACHINE LEARNING MODELS,” which is hereby incorporated by reference in its entirety as if it were fully set forth herein.

### BACKGROUND

Every day, millions of people are diagnosed with a wide variety of medical conditions, ranging greatly in type and severity. A patient who has been diagnosed with a medical condition often experiences many hardships as a result of their diagnosis. In addition to physical effects, such as pain, discomfort, or loss of mobility that may accompany the diagnosis, the hardships faced by patients often further include financial difficulties resulting from lost work, medical bills and the cost of treatments. Further still, a patient's diagnosis often negatively impacts their social interactions, quality of life, and overall emotional well-being. The result is that many patients experience significant psychological distress, and often do not receive effective support or treatment to alleviate this distress.

Additionally, psychological distress often exacerbates the physical symptoms associated with a patient's diagnosis, which in turn can lead to even greater psychological distress. As one specific example, symptoms associated with a gastrointestinal (GI) disorder, such as irritable bowel syndrome (IBS), are often triggered by stress, and psychological issues such as depression and/or anxiety can worsen those symptoms. Often, when a patient is diagnosed with one or more medical conditions, the patient is referred to additional health practitioners for further care and treatment. For example, a patient who has been diagnosed with a gastrointestinal (GI) disorder may be referred to a psychologist, psychiatrist, counselor, or other mental health practitioner to address any psychological issues, such as stress, anxiety, and/or depression that may stem from the diagnosis.

Health practitioners such as these typically utilize one or more techniques, methodologies, and/or modalities, such as, but not limited to, psychotherapy, cognitive behavioral therapy (CBT), acceptance commitment therapy (ACT), dialectical behavioral therapy (DBT), exposure therapy, mindfulness-based cognitive therapy (MCBT), hypnotherapy, experiential therapy, and psychodynamic therapy, to assist patients with management of their physiological and/or psychological conditions. In some embodiments, components of the above-listed therapeutic modalities may be combined to tailor a therapy regimen to the needs of a particular patient.

Even though a therapy or a combination of therapies can be implemented in a variety of ways, common elements can be identified. As one illustrative example, CBT is typically administered utilizing a set of structured sessions or modules, which are each designed to teach a patient skills that enable the patient to better understand and manage thoughts and behaviors that may be negatively affecting their mental and physical states. Although aspects of CBT may be fairly structured, there is still enough flexibility to allow CBT to be adapted for use in treatment of particular conditions. One such example is that of utilizing CBT to treat IBS. The rationale for applying CBT to treat IBS is grounded in the biopsychosocial model, which states that one's biology, thoughts, emotions, external events, and behaviors influence IBS symptom expression in a bidirectional way. In addition to IBS, psychological and/or behavioral therapies may be utilized to treat a wide variety of health conditions, as will be discussed herein.

A behavioral therapy, such as CBT, may be administered to patients across a range of delivery modalities. For example, the therapy may be administered by a health practitioner in-person, individually, or in-person, in a group. Alternatively, the therapy may be administered remotely, such as telephonically, over the internet, or through a computer software application. Further, a therapy may be administered to a patient without the direct involvement of a health practitioner, for example, a therapy may be administered to a patient remotely by a software application and/or may be self-administered by the patient. For any given therapy, regardless of delivery modality, one or more therapeutic protocols are typically defined for administration of the therapy, wherein the therapeutic protocols govern the manner in which the therapy is administered to a patient. For example, a therapy may include a series of lessons, questionnaires, and exercises, and a related protocol may dictate the order, speed, and/or frequency in which various lessons, exercises and questionnaires are presented to a patient. A protocol may also dictate the specific layout, content and general presentation of the various lessons, exercises and questionnaires. A protocol can be as specific as to dictate each word or sequence of words selected for use in the therapy. A therapy may be administered to a patient according to any number of protocols or any number of combinations of protocols.

Upon administration of a therapy to a patient according to a particular protocol or combination of protocols, data may be generated and/or collected regarding the effectiveness of the therapy and/or the associated protocols. Upon a determination that a particular protocol is not effective in treating a specific patient, group of patients, or medical condition, the protocol may be adjusted in an effort to find a more effective manner in which to administer the therapy. As one simplified example, a current therapeutic protocol may dictate that a series of questions should be asked to the patient in the order “A, B, C.” After administration of the therapy, it may be determined that the protocol of asking the questions in the order “A, B, C” was not an effective protocol, in which case the protocol might be adjusted to instead ask the questions in the order “C, B, A.”

Unfortunately, due to the vast quantity of data related to therapies, protocols, combinations of protocols, and associated protocol effectiveness data, the task of determining which protocols are effective and which are not, and further determining how to adjust the protocols to maximize effectiveness, becomes a monumental task. The problem is further compounded when you take into account that while certain protocols may be effective for one type of patient, the same protocols may not be effective for other types of patients, and so the protocols need to be tailored to particular patient characteristics and/or cultures associated with the patients in order to be maximally effective.

As one specific illustrative example, the effectiveness of the language used in therapeutic protocols varies greatly from culture to culture. A protocol that is effective for patients in English speaking cultures may be ineffective for patients in non-English speaking cultures. In some cases, a protocol that is acceptable for patients in English speaking cultures may actually be offensive to patients in non-English speaking cultures. Further, the effectiveness of various protocols may also be dependent on a regional sub-culture associated with the patient. For example, a protocol that is effective for a patient who lives in California may not be effective for a patient that lives in Texas. Further still, the effectiveness of various protocols may be dependent on the generational culture associated with the patient. For example, a protocol that is effective for a baby boomer may not be effective for a millennial.

Thus, especially when taking cultural and language differences into account, the result is that there are thousands or millions of possible protocols or combinations of protocols that need to be translated and analyzed in light of patient data associated with thousands or millions of patients, as well as in light of cultural sensitivity data associated with hundreds or thousands of cultures, in order to provide the most effective treatment to patients associated with a wide range of cultures. Clearly, this task is not feasible for a single human being or even a group of human beings to complete, even given unlimited time and resources.

Further, a large amount of data regarding protocol effectiveness for patients associated with a first culture may already exist, while there may be very little data available regarding protocol effectiveness for patients associated with cultures other than the first culture. Thus, absent the method and system disclosed herein, the process of generating maximally effective therapeutic protocols for patients associated with cultures other than the first culture may take a great deal more time than generating maximally effective therapeutic protocols for patients associated with the first culture.

Therefore dynamically, efficiently, and rapidly generating culturally sensitive therapeutic protocols presents a technical problem, which requires a technical solution. As software applications continue to replace human interactions, this problem becomes even more pronounced, as people are increasingly relying on applications to provide them with support and assistance in a wide variety of aspects of their daily lives. This is especially true in times of global crises, such as the 2020 worldwide pandemic, which has limited the availability and/or desirability of in-person medical appointments. When administering a therapy remotely, for example, over the internet, through a website, or through a software application, the protocols utilized are traditionally statically programmed into the software and thus are not able to be readily modified when new data, such as data relating to the effectiveness of the protocols, is received. Thus, due to the large number of people diagnosed with medical conditions, and the increasing demand for remote administration of therapies, the failure of traditional solutions to address the problem of dynamically, efficiently, and rapidly generating culturally sensitive therapeutic protocols to ensure that patients associated with a wide range of cultures receive effective care, support, and treatment, has the potential to lead to significant consequences for a large number of people.

What is needed, therefore, is a technical solution to the technical problem of dynamically, efficiently, and rapidly generating culturally sensitive therapeutic protocols to ensure that patients associated with a wide range of cultures receive effective care, support, and treatment.

### SUMMARY

Embodiments of the present disclosure provide a technical solution to the technical problem of dynamically, efficiently, and rapidly generating culturally sensitive therapeutic protocols to ensure that patients associated with a wide range of cultures receive effective care, support, and treatment. In the disclosed embodiments, when a patient associated with a first culture has been diagnosed with one or more health conditions, an appropriate therapy is selected for administration to the patient, depending on the particular diagnosis. In many instances, the therapy selected may be a psychological therapy that is intended to treat psychological issues related to the patient's diagnosis. In some embodiments, psychological therapies may include behavioral therapies. As used herein the term “therapy,” “psychological therapy,” “behavioral therapy,” or “therapeutic modality” may include psychological techniques, methodologies, and/or modalities utilized to treat patients, such as, but not limited to psychotherapy, cognitive behavioral therapy (CBT), acceptance commitment therapy (ACT), dialectical behavioral therapy (DBT), exposure therapy, mindfulness-based cognitive therapy (MCBT), hypnotherapy, experiential therapy, and psychodynamic therapy. In some embodiments, components of the above-listed therapies may be combined to tailor a therapy regimen to the needs of a particular patient.

In one embodiment, once an appropriate psychological therapy or combination of therapies has been selected for a patient associated with a first culture, historical therapeutic protocols associated with the first culture are provided to a protocol generation system associated with the first culture.

In one embodiment, once historical therapeutic protocols associated with a first culture have been provided to a protocol generation system associated with the first culture, the protocol generation system associated with the first culture is utilized to generate one or more therapeutic protocols that are predicted to be maximally effective for patients associated with the first culture.

As used herein, the term “protocol” or “therapeutic protocol” may include procedures and/or systems of rules for administration of a psychological therapy. A therapeutic protocol defines the rules, syntax, semantics, and synchronization of communications between a patient and the party that is administering the therapy. As noted above, a therapy being administered to a patient may include various lessons, exercises, and questionnaires. An associated therapeutic protocol, therefore, may dictate the order, speed, and/or frequency in which the various lessons, exercises and questionnaires are presented to a patient. A protocol may also dictate the specific content, layout, presentation, and word sequences selected for incorporation into the various lessons, exercises and questionnaires. As used herein, the term “historical therapeutic protocol” may include protocols that have previously been generated, tested, established, and/or clinically validated for use in administration of a therapy.

In one embodiment, generating one or more therapeutic protocols that are maximally effective for a patient associated with the first culture includes administering the psychological therapy to the patient according to one or more historical therapeutic protocols. As used herein, the phrase “administration of a therapy” may include administration of a therapy to a patient by a health practitioner, or administration of a therapy to a patient without the direct involvement of a health practitioner.

In one embodiment, as the psychological therapy is being administered to the patient, the patient's responses to the historical therapeutic protocols are monitored to obtain patient protocol response data. Patient protocol response data may also be collected after administration of the therapy. As used herein, in various embodiments, “patient response data” or “patient protocol response data” may include direct verbal or written feedback from the patient, indirect feedback, such as an indication of whether a particular therapeutic protocol appears to be having an effect on the patient, and/or other measureable data such as, but not limited to, physiological sensor data, and/or click-stream data showing patient engagement with the content of the therapy.

In one embodiment, the patient protocol response data is analyzed to determine the effectiveness of the therapeutic protocols administered to the patient as part of the therapy, and patient protocol effectiveness data is generated representing the effectiveness of the therapeutic protocols for the patient. In one embodiment, the patient protocol effectiveness data and patient data associated with the patient are analyzed to generate one or more patient profiles. As used herein, the term “patient data” may include data associated with the patient, such as, but not limited to age, sex, ethnicity, marital status, income level, geographic location, personal and family medical history, including the current medical issue that the therapy is designed to treat. As used herein, the term “patient profile” may include models or templates that describe a particular type of patient.

In one embodiment, historical therapeutic protocol data is correlated with patient profile data to generate therapeutic protocol effectiveness model training data, which is used as training data to train one or more machine learning based models. In one embodiment, the machine learning based models are models that predict the effectiveness of a given therapeutic protocol, and training the models with the therapeutic protocol effectiveness model training data results in the creation of one or more trained therapeutic protocol effectiveness prediction models. In various embodiments, the above described process may continue indefinitely, or may be terminated at any time at the discretion of an administrator of the method and system disclosed herein.

In various embodiments, once the therapeutic protocol effectiveness prediction models are trained, they can be used in a variety of ways. In one embodiment, the trained therapeutic protocol effectiveness prediction models can be used to dynamically generate one or more improved or maximally effective therapeutic protocols for a specific patient, or a specific type of patient associated with the first culture. In other embodiments, the trained therapeutic protocol effectiveness prediction models can be utilized independently of a specific patient, for example, to generate one or more improved or maximally effective therapeutic protocols, which may be determined to be generally effective for patients associated with the first culture, regardless of the patient's individual characteristics, background, and history.

In one embodiment, in the case of dynamically generating one or more improved or maximally effective therapeutic protocols for a specific patient associated with the first culture, a psychological therapy is selected for administration to the patient. Patient data associated with the patient and patient profile data associated with the predefined patient profiles are analyzed to select a patient profile that is the best match for the specific patient. In one embodiment, the selected patient profile data is provided to the trained therapeutic protocol effectiveness prediction models associated with the first culture.

In one embodiment, new therapeutic protocol test data, representing one or more new therapeutic protocols associated with the psychological therapy, is generated or otherwise obtained, wherein the new therapeutic protocols are new protocols to be considered for use in administration of the psychological therapy. In one embodiment, the new therapeutic protocol test data is provided to the trained therapeutic protocol effectiveness prediction models. In one embodiment, the trained therapeutic protocol effectiveness prediction models are utilized to generate predicted protocol effectiveness data for the new protocols represented by the new therapeutic protocol test data. In one embodiment, the predicted protocol effectiveness data associated with the new therapeutic protocols, and historical protocol effectiveness data associated with historical therapeutic protocols is analyzed to determine and select one or more effective therapeutic protocols.

In one embodiment, once one or more effective therapeutic protocols have been selected, effective protocol definition data associated with the one or more effective therapeutic protocols is utilized to generate one or more maximally effective therapeutic protocols for use in administration of the selected psychological therapy. In one embodiment, maximally effective protocol definition data associated with the one or more maximally effective therapeutic protocols is incorporated into historical protocol definition data for future use in administration of a psychological therapy. This allows the maximally effective therapeutic protocols to be later used in the generation of model training data, so that the therapeutic protocol effectiveness prediction models can be continually trained with new data. In one embodiment, the selected psychological therapy is then administered to the patient according to the maximally effective therapeutic protocols.

In one embodiment, in the case of generating an improved or maximally effective therapeutic protocol that is generally effective for patients, a process similar to that described above may be utilized, without providing patient-specific profile data to the trained therapeutic imagery effectiveness prediction models. For example, in some embodiments, a psychological therapy is selected for administration to one or more patients, new therapeutic protocol test data is generated and provided to the trained therapeutic protocol effectiveness prediction models, and the trained therapeutic protocol effectiveness prediction models are utilized to generate predicted protocol effectiveness data for the new protocols. In some embodiments, the predicted protocol effectiveness data and the historical protocol effectiveness data are analyzed to select one or more effective therapeutic protocols. Protocol definition data associated with the one or more effective therapeutic protocols is then utilized to generate one or more maximally effective therapeutic protocols. In one embodiment, maximally effective protocol definition data associated with the one or more maximally effective therapeutic protocols is incorporated into historical protocol definition data for future use in administration of a psychological therapy.

The above described processes result in generation of one or more therapeutic protocols that are predicted to be maximally effective for patients associated with the first culture, which may then be administered to a patient associated with the first culture, thus ensuring that the patient receives effective care, support, and treatment. Further, the above machine learning process employs a feedback loop, such that the therapeutic effectiveness prediction models can be dynamically refined to account for newly received effectiveness data, thus improving the accuracy of the effectiveness predictions generated by the models.

In one embodiment, once one or more maximally effective protocols are generated for patients associated with the first culture, the one or more first culture maximally effective protocols are translated from a language and/or dialect associated with the first culture to a language and/or dialect associated with one or more cultures other than the first culture. In one embodiment, cultural sensitivity data associated with the one or more cultures other than the first culture is obtained and utilized to determine whether one or more modifications should be made to the translated therapeutic protocols.

In one embodiment, upon a determination that one or more modifications should be made to the translated therapeutic protocols, the cultural sensitivity data is utilized to transform the one or more translated therapeutic protocols into protocols that are culturally sensitive to the one or more cultures other than the first culture. In one embodiment, the culturally sensitive protocols associated with each of the one or more cultures are provided to a protocol generation system associated with each of the one or more cultures other than the first culture, and the protocol generation systems associated with each of the one or more cultures other than the first culture are utilized to generate one or more therapeutic protocols that are predicted to be maximally effective for patients associated with each of the one or more cultures other than the first culture.

In one embodiment, generating one or more therapeutic protocols that are predicted to be maximally effective for patients associated with each of the one or more cultures includes the same operations described above with respect to the generation of protocols that are predicted to be maximally effective for patients associated with the first culture.

As a result of these and other disclosed features, which are discussed in more detail below, the disclosed embodiments provide an effective and efficient technical solution to the technical problem of dynamically, efficiently, and rapidly generating culturally sensitive therapeutic protocols to ensure that patients associated with a wide range of cultures receive effective care, support, and treatment.

### BRIEF DESCRIPTION OF THE DRAWINGS

FIG. 1 is a block diagram of a system for generating culturally sensitive therapeutic protocols, in accordance with one embodiment.

FIG. 2A is a block diagram of a training environment for creating trained therapeutic protocol effectiveness prediction models, in accordance with one embodiment.

FIG. 2B and FIG. 2C are diagrams illustrating components of a therapy and the application of various therapeutic protocols to the therapy components, in accordance with one embodiment.

FIG. 3 is a block diagram of a runtime environment for utilizing trained therapeutic protocol effectiveness prediction models to generate maximally effective therapeutic protocols for a specific patient, in accordance with one embodiment.

FIG. 4 is a block diagram of a runtime environment for utilizing trained therapeutic protocol effectiveness prediction models to generate generalized maximally effective therapeutic protocols, in accordance with one embodiment.

FIG. 5 is a flow chart of a process for generating culturally sensitive therapeutic protocols, in accordance with one embodiment.

FIG. 6 is a flow chart of a process for creating trained therapeutic protocol effectiveness prediction models, in accordance with one embodiment.

FIG. 7 is a flow chart of a process for utilizing trained therapeutic protocol effectiveness prediction models to generate maximally effective therapeutic protocols for a specific patient, in accordance with one embodiment.

FIG. 8 is a flow chart of a process for utilizing trained therapeutic protocol effectiveness prediction models to generate generalized maximally effective therapeutic protocols, in accordance with one embodiment.

Common reference numerals are used throughout the figures and the detailed description to indicate like elements. One skilled in the art will readily recognize that the above figures are merely illustrative examples and that other architectures, modes of operation, orders of operation, and elements/functions can be provided and implemented without departing from the characteristics and features of the invention, as set forth in the claims.

### DETAILED DESCRIPTION

Embodiments will now be discussed with reference to the accompanying figures, which depict one or more exemplary embodiments. Embodiments may be implemented in many different forms and should not be construed as limited to the embodiments set forth herein, shown in the figures, or described below. Rather, these exemplary embodiments are provided to allow a complete disclosure that conveys the principles of the invention, as set forth in the claims, to those of skill in the art.

### Term Definitions

As used herein, the term “patient” or “participant” may include an individual who has been diagnosed with one or more health conditions, an individual who is the recipient of a therapy in a clinical or non-clinical setting, and/or an individual who has not been diagnosed with a health condition, but is a recipient of a therapy in a clinical or non-clinical setting. Therefore, although the term “patient” will be used commonly throughout the enclosed specification, the term “participant” may also be used to indicate that applications of the methods and systems disclosed herein, used outside of a clinical setting, are also contemplated by the following disclosure.

As used herein, the term “culture” or “cultural group” may include any group of people who share common characteristics, such as, but not limited to, language, customs, conventions, and values. A “culture” or “cultural group” can be defined in a variety of ways, such as, but not limited to, a culture based on the geographical location and/or origin of a group of people (e.g. Western culture vs. Eastern culture), a culture based on religion (e.g. Christian culture vs. Muslim culture), and/or a culture based on age group (e.g. Baby boomer culture vs. Millennial culture).

As used herein the term “therapy,” “psychological therapy,” “behavioral therapy,” or “therapeutic modality” may include psychological techniques, methodologies, and/or modalities utilized to treat patients, such as, but not limited to psychotherapy, cognitive behavioral therapy (CBT), acceptance commitment therapy (ACT), dialectical behavioral therapy (DBT), exposure therapy, mindfulness-based cognitive therapy (MCBT), hypnotherapy, experiential therapy, and psychodynamic therapy. In some embodiments, components of the above-listed therapies may be combined to tailor a therapy regimen to the needs of a particular patient.

As used herein, the phrase “administration of a therapy” or “administering a therapy” may include providing, delivering, and/or applying a therapy to a patient. A therapy may be administered to a patient directly by a health practitioner. A therapy may be administered to a patient remotely, for example, over the internet or by computer software, without the direct involvement of a health practitioner. For example, the therapy may be self-administered by the patient. A therapy may also be administered to a patient remotely with partial involvement of a health practitioner. For example, the therapy to be administered may be selected by a health practitioner, but the therapy may then be self-administered by the patient, utilizing computer software, or the therapy may be administered to the patient by computer software, but a health practitioner may monitor the patient's response data.

As used herein, the term “protocol” or “therapeutic protocol” may include procedures and/or systems of rules for administration of a psychological therapy. A therapeutic protocol defines the rules, syntax, semantics, and synchronization of communications with a patient. For example, a therapy may include a series of lessons, questionnaires, and exercises, and a related protocol may dictate the order, speed, and/or frequency in which various lessons, exercises and questionnaires are presented to a patient. A protocol may also dictate the specific layout, content and general presentation of the various lessons, exercises and questionnaires. A protocol can be as specific as to dictate each word or sequence of words selected for use in the therapy. A therapy may be administered to a patient according to any number of protocols or any number of combinations of protocols.

As used herein, the terms “current therapeutic protocol” or “historical therapeutic protocol” may include protocols that have previously been generated, tested, established, and/or clinically validated for use in administration of a therapy.

As used herein, the terms “new protocol,” and “new therapeutic protocol” may include protocols that have not been previously generated, tested, established, and/or clinically validated for use in administration of a therapy. Additionally, the terms “new protocol,” and “new therapeutic protocol” may also include protocols that have been previously generated and/or tested, but may not yet be established and/or clinically validated for use in administration of a therapy. In various embodiments, new therapeutic protocols may also include potential therapeutic protocols or candidate therapeutic protocols, in the sense that they are protocols that are being considered for use in a therapy.

As used herein, the terms “improved protocol” or “improved therapeutic protocol” may include new therapeutic protocols that have been found to be more effective than a current or historical therapeutic protocol when used in a therapy to treat one or more patients, wherein effectiveness of a particular therapeutic protocol is determined by a variety of clinically validated outcome measures, as will be discussed in additional detail below.

As used herein, the terms “maximally effective protocol” or “maximally effective therapeutic protocol” may include therapeutic protocols that have been determined to be the most effective therapeutic protocols, for a particular period of time, out of the new, current, and/or historical therapeutic protocols of comparable type, wherein effectiveness of a particular therapeutic protocol is determined by a variety of clinically validated outcome measures, as will be discussed in additional detail below. An improved therapeutic protocol and/or a maximally effective therapeutic protocol may be the most effective, during the particular period of time, for the general patient population, the most effective for a particular group of patients, and/or the most effective for a specific individual patient, and the system and method disclosed herein accounts for these differences according to predefined guidelines, as will be discussed in further detail below.

As used herein, the terms “culturally sensitive translation,” “culturally sensitive protocol,” or “culturally sensitive translated protocol” may include therapeutic protocols that take into account the language, customs, conventions, and values of a culture or cultural group when translating a therapeutic protocol from a language and/or dialect associated with a first culture to a language and/or dialect associated with a culture other than the first culture, such that the translated protocol is likely to be acceptable, inoffensive, and/or effective for patients associated with the culture other than the first culture.

As used herein, in various embodiments, the terms “patient response data” or “patient protocol response data” may include direct verbal or written feedback from the patient, indirect feedback, such as an indication of whether a particular therapeutic protocol appears to be having an effect on the patient, and/or other measureable data such as, but not limited to, physiological sensor data and/or click-stream data showing patient engagement with the content of the therapy.

As used herein, the term “patient data” may include data associated with a patient, such as, but not limited to age, sex, ethnicity, marital status, income level, geographic location, personal and family medical history, including the current medical issue that the therapy is designed to treat.

As used herein, the term “patient profile” may include models or templates that describe a particular type of patient.

### System

Embodiments of the present disclosure provide a technical solution to the technical problem of dynamically, efficiently, and rapidly generating culturally sensitive therapeutic protocols to ensure that patients associated with a wide range of cultures receive effective care, support, and treatment. In the disclosed embodiments, a protocol generation system for a first culture takes historical therapeutic protocol data associated with the first culture as input data, provides the historical therapeutic protocol data associated with the first culture to a protocol effectiveness prediction training environment, which trains therapeutic protocol effectiveness prediction models to predict the effectiveness of a variety of therapeutic protocols for patients associated with the first culture. The trained therapeutic protocol effectiveness prediction models associated with the first culture are then incorporated into an effective protocol generation runtime environment, which is utilized to generate therapeutic protocols that are predicted to be maximally effective for patients associated with the first culture.

In one embodiment, the therapeutic protocols that are predicted to be maximally effective for patients associated with the first culture are provided to a protocol translation module, which translates the therapeutic protocols that are predicted to be maximally effective for patients associated with the first culture from a language and/or dialect associated with the first culture, to a language and/or dialect associated with one or more cultures other than the first culture, resulting in translated protocol data for the one or more cultures other than the first culture. In one embodiment the translated protocol data is provided to a culturally sensitive protocol translation module, which utilizes cultural sensitivity data associated with one or more cultures to determine whether modifications should be made to the translated therapeutic protocols to adjust for cultural sensitivities. Upon a determination that one or more modifications should be made, the culturally sensitive protocol translation module utilizes the cultural sensitivity data to transform the translated therapeutic protocols into protocols that are culturally sensitive to the one or more cultures other than the first culture.

In one embodiment, similarly to the functioning of the protocol generation system for the first culture, individual protocol generation systems associated with each of the cultures represented by the culturally sensitive translated protocols utilize data associated with the culturally sensitive translated protocols to generate therapeutic protocols that are predicted to be maximally effective for patients associated with each of the cultures represented by the culturally sensitive translated protocols.

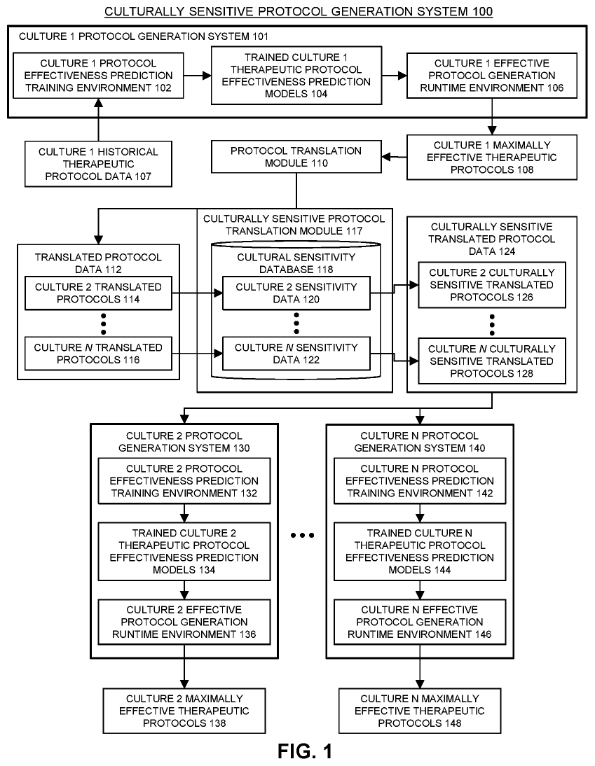

FIG. 1 is a block diagram of a system 100 for generating culturally sensitive therapeutic protocols, in accordance with one embodiment.

In various embodiments, culturally sensitive protocol generation system 100 includes culture 1 historical therapeutic protocol data 107, culture 1 protocol generation system 101, and culture 1 maximally effective therapeutic protocols 108. In one embodiment, culture 1 protocol generation system 101 includes culture 1 protocol effectiveness prediction training environment 102, trained culture 1 therapeutic protocol effectiveness prediction models 104, and culture 1 effective protocol generation runtime environment 106.

In various embodiments, culturally sensitive protocol generation system 100 further includes protocol translation module 110, translated protocol data 112, culturally sensitive protocol translation module 117, and culturally sensitive translated protocol data 124. In one embodiment, translated protocol data 112 includes culture 2 translated protocols 114 through culture n translated protocols 116. In one embodiment, culturally sensitive protocol translation module 117 includes cultural sensitivity database 118, which further includes culture 2 sensitivity data 120 through culture n sensitivity data 122. In one embodiment, culturally sensitive translated protocol data 124 includes culture 2 culturally sensitive translated protocols 126 through culture n culturally sensitive translated protocols 128.

In various embodiments, culturally sensitive protocol generation system 100 further includes culture 2 protocol generation system 130 through culture n protocol generation system 140, and culture 2 maximally effective therapeutic protocols 138 through culture n maximally effective therapeutic protocols 148. In one embodiment, culture 2 protocol generation system 130 includes culture 2 protocol effectiveness prediction training environment 132, trained culture 2 therapeutic protocol effectiveness prediction models 134, and culture 2 effective protocol generation runtime environment 136. In one embodiment, culture n protocol generation system 140 includes culture n protocol effectiveness prediction training environment 142, trained culture n therapeutic protocol effectiveness prediction models 144, and culture n effective protocol generation runtime environment 146. Each of the above listed elements will be discussed in additional detail below.

As will be discussed in further detail below, culturally sensitive protocol generation system 100 utilizes historical therapeutic protocol data associated with a first culture to dynamically, efficiently, and rapidly generate culturally sensitive therapeutic protocols for any number of different cultures. In one embodiment the historical therapeutic protocol data is protocol data that is associated with a therapy to be administered to a patient. In the illustrative embodiment of FIG. 1, culture 1 protocol generation system 101 takes culture 1 historical therapeutic protocol data 107 as input data, provides culture 1 historical therapeutic protocol data 107 to culture 1 protocol effectiveness prediction training environment 102, which generates trained culture 1 therapeutic protocol effectiveness prediction models 104. Trained culture 1 therapeutic protocol effectiveness prediction models 104 are then incorporated into culture 1 effective protocol generation runtime environment 106, which is utilized to generate culture 1 maximally effective therapeutic protocols 108.

In one embodiment, culture 1 maximally effective therapeutic protocols 108 are provided to protocol translation module 110, which translates culture 1 maximally effective therapeutic protocols 108 from a language and/or dialect associated with the first culture, to a language and/or dialect associated with one or more cultures other than the first culture, resulting in translated protocol data 112, which, in the illustrative embodiment of FIG. 1, includes culture 2 translated protocols 114 through culture n translated protocols 116. In one embodiment translated protocol data 112 is provided to culturally sensitive protocol translation module 117, which utilizes cultural sensitivity data associated with one or more cultures, such as culture 2 sensitivity data 120 through culture n sensitivity data 122 of cultural sensitivity database 118, to determine whether modifications should be made to the translated protocol data 112 to adjust for cultural sensitivities. Upon a determination that one or more modifications should be made to the translated protocol data 112, culturally sensitive protocol translation module 117 utilizes culture 2 sensitivity data 120 through culture n sensitivity data 122 of cultural sensitivity database 118 to transform the translated protocol data 112 into culturally sensitive translated protocol data 124, which, in the illustrative embodiment of FIG. 1, includes culture 2 culturally sensitive translated protocols 126 through culture n culturally sensitive translated protocols 128.

Similarly to the functioning of culture 1 protocol generation system 101, in various embodiments, culture 2 protocol generation system 130 utilizes culture 2 culturally sensitive translated protocol data 126 to generate culture 2 maximally effective therapeutic protocols 138, wherein culture 2 maximally effective therapeutic protocols 138 are protocols that have been determined by culture 2 protocol generation system 130 to be maximally effective for patients associated with culture 2. Likewise culture n protocol generation system 140 utilizes culture n culturally sensitive translated protocol data 128 to generate culture n maximally effective therapeutic protocols 148, wherein culture n maximally effective therapeutic protocols 148 are protocols that have been determined by culture n protocol generation system 140 to be maximally effective for patients associated with culture n.

In one embodiment, culture 1 historical therapeutic protocol data 107 represents historical therapeutic protocols associated with a first culture. In one embodiment, culture 1 historical therapeutic protocol data 107 is provided to a protocol generation system associated with the first culture, such as culture 1 protocol generation system 101. In one embodiment, culture 1 protocol generation system 101 is utilized to generate one or more therapeutic protocols that are maximally effective for patients associated with the first culture, such as culture 1 maximally effective therapeutic protocols 108. As noted above, in one embodiment, culture 1 protocol generation system 101 includes culture 1 protocol effectiveness prediction training environment 102.

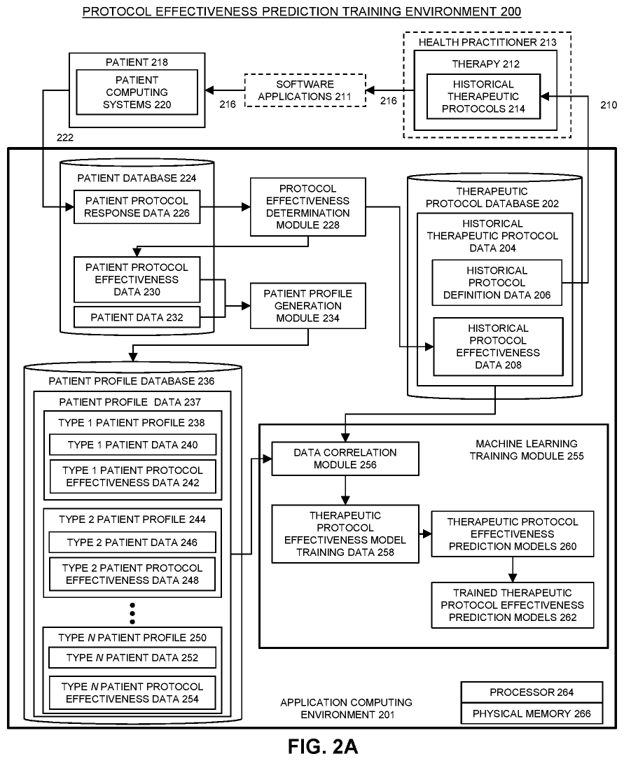

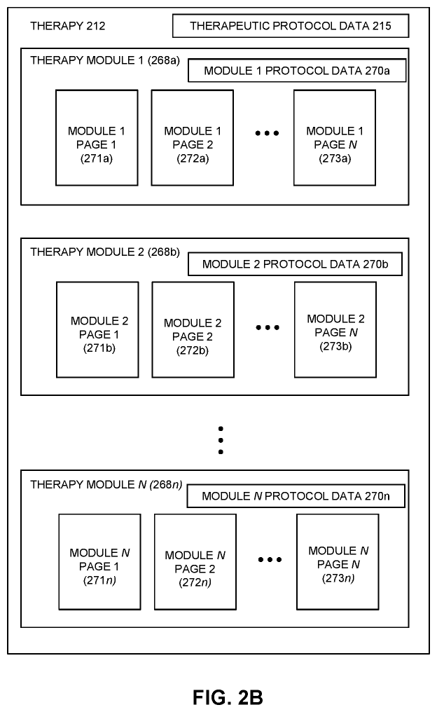

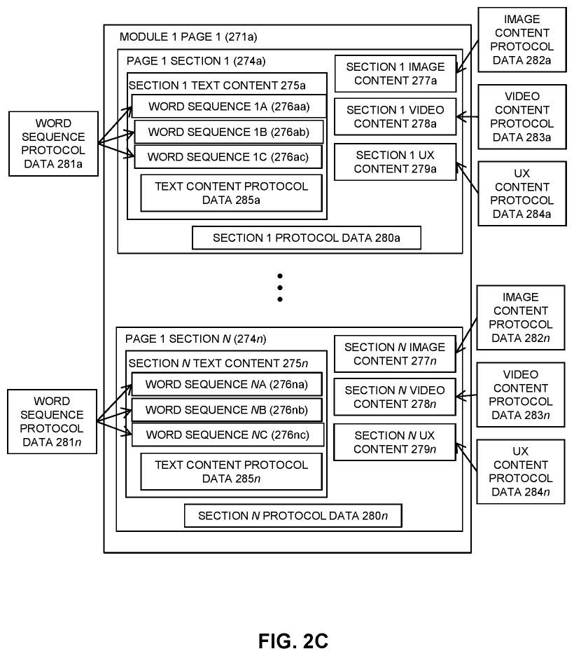

FIG. 2A is a block diagram of a protocol effectiveness prediction training environment 200 for creating trained therapeutic protocol effectiveness prediction models 262, in accordance with one embodiment.

Referring now to FIG. 1 and FIG. 2A together, in one embodiment, protocol effectiveness prediction training environment 200 operates independently of the culture that protocols are being generated for. For example, protocol effectiveness prediction training environment 200 is capable of generating trained therapeutic protocol effectiveness prediction models that are specific to a first culture, a second culture, or any number of differing types of cultures. The key difference lies in the historical protocol data that is ingested by the system. For example, in one embodiment, historical therapeutic protocols associated with a first culture, represented by culture 1 historical therapeutic protocols 107 of FIG. 1, are provided to protocol effectiveness prediction training environment 200 of FIG. 2. Thus, the protocol effectiveness prediction training environment 200 that operates on culture 1 historical therapeutic protocol data 107 is referred to in FIG. 1 as culture 1 protocol effectiveness prediction training environment 102, which is part of culture 1 protocol generation system 101. In the illustrative embodiment of FIG. 2, the culture 1 historical therapeutic protocol data 107 is represented by the historical therapeutic protocol data 204, which will be discussed in greater detail below.

Referring now specifically to FIG. 2, in various embodiments, protocol effectiveness prediction training environment 200 includes application computing environment 201, health practitioner 213, software applications 211, therapy 212 and associated historical therapeutic protocols 214, patient 218 and associated patient computing systems 220. In one embodiment, protocol effectiveness prediction training environment 200 further includes communications channel 210, which facilitates retrieval of data from application computing environment 201 to be incorporated into therapy 212, communications mechanisms 216, which facilitates administration of therapy 212 to patient 218, and communications channel 222, which facilitates transmission of data from patient 218 to application computing environment 201. Each of the above listed elements will be discussed in further detail below.

In various embodiments, application computing environment 201 includes therapeutic protocol database 202, patient database 224, and patient profile database 236. In one embodiment, therapeutic protocol database 202 includes historical therapeutic protocol data 204, which further includes historical protocol definition data 206, and historical protocol effectiveness data 208. In one embodiment, patient database 224 includes patient protocol response data 226, patient protocol effectiveness data 230, and patient data 232. In one embodiment, patient profile database 236 includes patient profile data 237, which further includes type 1 patient profile 238, type 2 patient profile 244 through type n patient profile 250. In one embodiment, type 1 patient profile 238 further includes type 1 patient data 240 and type 1 patient protocol effectiveness data 242, type 2 patient profile 244 further includes type 2 patient data 246 and type 2 patient protocol effectiveness data 248, and type n patient profile 250 further includes type n patient data 252 and type n patient protocol effectiveness data 254. Each of the above listed elements will be discussed in further detail below.

In various embodiments, application computing environment 201 further includes several process modules, such as protocol effectiveness determination module 228, patient profile generation module 234, and machine learning training module 255. In one embodiment, machine learning training module 255 further includes data correlation module 256, therapeutic protocol effectiveness model training data 258, therapeutic protocol effectiveness prediction models 260, and trained therapeutic protocol effectiveness prediction models 262. In one embodiment, application computing environment 201 further includes processor 264 and physical memory 266, which together coordinate the operation and interaction of the data and data processing modules associated with application computing environment 201. Each of the above listed elements will be discussed in further detail below.

In one embodiment, patient 218 is a patient who has been diagnosed with a medical condition, and a determination is made as to whether patient 218 will benefit from receiving one or more therapies. In one embodiment, the determination is made by health practitioner 213. In one embodiment, the determination is made by patient 218. In one embodiment, the determination is made by computer software algorithms. In various other embodiments, the determination may be made by one or more third parties. As one specific example, symptoms associated with a gastrointestinal (GI) health condition, such as irritable bowel syndrome (IBS), are often triggered by stress, and psychological issues such as, but not limited to, stress, anxiety, and depression, can worsen those symptoms. Patients who are suffering from stress, anxiety, and/or depression related to their medical diagnosis often benefit from receiving certain types of psychological therapies to help them better understand and manage their physiological and/or psychological conditions. Examples of therapeutic modalities that may be beneficial to patients include, but are not limited to, psychotherapy, cognitive behavioral therapy (CBT), acceptance commitment therapy (ACT), dialectical behavioral therapy (DBT), exposure therapy, mindfulness-based cognitive therapy (MCBT), hypnotherapy, experiential therapy, and psychodynamic therapy. In some embodiments, components of the above-listed therapies may be combined to tailor a therapy regimen to the needs of a particular patient.

While GI conditions, such as irritable bowel syndrome (IBS), gastroesophageal reflux disease (GERD), and inflammatory bowel disease (IBD), are specifically indicated throughout the present disclosure, in various embodiments, one or more of the above-listed therapies may be utilized to treat a variety of other health conditions, such as, but not limited to, fibromyalgia, endometriosis, vulvodynia, cystitis, chronic itch, chronic cough, chronic migrate, encopresis, and constipation.

In some embodiments the therapies discussed herein may also be used to treat lactose intolerance, ulcers (e.g., peptic ulcer disease, gastric ulcers, etc.), functional dyspepsia, hernias, celiac disease, diverticulitis, malabsorption, short bowel syndrome, intestinal ischemia, pancreatitis, cysts, gastroparesis, gastritis, esophagitis, achalasia, strictures, anal fissures, hemorrhoids, proctitis, prolapse, gall stones, cholecystitis, cholangitis, GI-associated cancers, bleeding, bloating, diarrhea, heartburn, fecal incontinence/encopresis, nausea, cyclic vomiting syndrome, abdominal pain, swallowing issues, weight maintenance issues, diabetes, cardiovascular diseases (e.g., hypertension), liver diseases, arthritis and joint diseases (e.g., rheumatoid arthritis), various allergies, asthma, and chronic obstructive pulmonary disease (COPD).

In one embodiment, once a determination has been made that patient 218 is likely to benefit from a particular therapy, such as therapy 212, the therapy 212 may be administered to the patient using one or more communication mechanisms 216. In some embodiments, communication mechanisms 216 include health practitioner 213 conducting a physical in-person meeting with patient 218 to verbally guide patient 218 through the therapy 212. In other embodiments, communication mechanisms 216 include administering the therapy 212 to patient 218 remotely, for example through a website, or through one or more software applications 211 that can be executed from patient computing systems 220. In one embodiment, the therapy 212 may be administered to patient 218 directly by health practitioner 213. In one embodiment, therapy 212 may be administered to patient 218 remotely, without the direct involvement of health practitioner 213. For example, therapy 212 may be self-administered by patient 218. In one embodiment, therapy 212 may also be administered to patient 218 remotely with partial involvement of health practitioner 213. For example, therapy 212 may be selected for administration by health practitioner 213, but therapy 212 may then be self-administered by patient 218, utilizing software applications 211, or therapy 212 may be administered to patient 218 by software applications 211, but health practitioner 213 may monitor patient 218's response data.

In various embodiments, patient computing systems 220 may include, but are not limited to, a desktop computing system, a mobile computing system, a virtual reality computing system, a gaming computing system, a computing system that utilizes one or more Internet of Things (IoT) devices, and/or any other type of computing system discussed herein, known at the time of filing, developed/made available after the time of filing, or any combination thereof.

As noted above, there are a variety of established and/or clinically validated therapies that have been shown to provide benefit to patients, and administration of these clinically validated therapies are typically governed by a collection of therapeutic protocols associated with the particular therapy. As noted above, and as used herein, the term “protocol” or “therapeutic protocol” may include procedures and/or systems of rules for administration of a psychological therapy. A therapeutic protocol defines the rules, syntax, semantics, and synchronization of communications between a patient and the party that is administering the therapy. For example, a particular therapy being administered to a patient may include various modules that contain content such as lessons, exercises, and questionnaires. An associated therapeutic protocol, therefore, may dictate the order, speed, and/or frequency in which the various lessons, exercises and questionnaires are presented to a patient. A protocol may also dictate the specific layout, content and general presentation of the various lessons, exercises and questionnaires. A protocol can be as specific as to dictate each word or sequence of words selected for use in the therapy. For any given therapy, protocols can be defined and applied to the therapy as a whole, or to any individual component or sub-component of a therapy. A therapy may be administered to a patient according to any number of protocols or any number of combinations of protocols.

FIG. 2B and FIG. 2C are diagrams illustrating components of a therapy and the application of various therapeutic protocols to the therapy components, in accordance with one embodiment.

FIG. 2B is an illustrative example, according to one embodiment, depicting an overview of therapy components and associated protocols. In the illustrative example of FIG. 2B, therapy 212 is governed by associated therapeutic protocol data 215, which dictates the protocols to be applied to the entirety of therapy 212. For example, therapeutic protocol data 215 might include data such as, but not limited to, data indicating the number of modules in therapy 212, the order in which to present the modules to the patient, and the duration of time between the presentation of the modules to the patient. In the illustrative example of FIG. 2B, the therapeutic protocol data 215 indicates that therapy 212 should include N modules, and that the modules should be presented in sequential order from module 1 to module N. Therapeutic protocol data 215 may also dictate that each sequential module should be presented to the patient one week after the prior module has been completed.

As one specific example, administration of a cognitive behavioral therapy (CBT) is often divided into eight separate sessions, or modules, with each module being presented to the patient approximately one week apart from the previous module. As one example, a first module of a therapy, such as therapy module 1 (268a) of therapy 212, may be focused on providing education to the patient related to their medical condition and/or education regarding the therapy and its goals. A second module of a therapy, such as therapy module 2 (268b) of therapy 212, may involve having the patient complete a self-assessment regarding their thoughts, emotions, and behaviors, with the goal of helping the patient to develop an understanding of how the interaction between the patient's thoughts, emotions, and behaviors impact the patient's medical symptoms. A final module of a therapy, such as therapy module N (268n) of therapy 212, may be focused on helping the patient develop skills to assist in processing their emotions, developing long-term goals, and managing symptom flare-ups.

It should be noted that the above examples are given for illustrative purposes only and are not intended to limit the scope of the invention as disclosed and claimed herein. It should be apparent to one of skill in the art that any number of sessions or modules may be included as part of a therapy, and the goals of each module, the presentation sequence of the modules and/or any other aspect of the therapy may be dictated by the associated protocol data. As noted above, at a high level, therapeutic protocol data 215 governs therapy 212, and further, each of the modules present may be governed by individual module protocol data.

For example, in the illustrative embodiment of FIG. 2B, therapy module 1 (268a) is governed by module 1 protocol data 270a, therapy module 2 (268b) is governed by module 2 protocol data 270b, and therapy module N (268n) is governed by module N protocol data 270n. In various embodiments, the module protocol data dictates the protocols to be applied to the entirety of an individual module. In various embodiments, module protocol data might include data such as, but not limited to, data indicating the number of pages in the associated module, and the order in which to present the pages to the patient. Module protocol data may also contain textual data, such as the text that should make up the title of the associated module In the illustrative embodiment of FIG. 2B, the module 1 protocol data 270a indicates that therapy module 1 (268a) should include N pages, and that the pages should be presented in sequential order from page 1 to page N. In the illustrative example of FIG. 2B, therapy module 1 (268a) includes module 1 page 1 (271a), module 1 page 2 (272a), through module 1 page N (273a). Therapy module 2 (268b) includes module 2 page 1 (271b), module 2 page 2 (272b), through module 2 page N, (273b). Therapy module N (268n) includes module N page 1 (271n), module N page 2 (272n), through module N page N (273n).

It should be noted again that the examples given above are for illustrative purposes only and are not intended to limit the scope of the invention as disclosed and claimed herein.

FIG. 2C is an illustrative example, according to one embodiment, depicting a detailed view of individual therapy module page components and associated protocols. For example, in the illustrative example of FIG. 2C, Module 1 page 1 (271a) includes several sections, such as page 1 section 1 (274a) through page 1 section N (274n). Similar to the description above with respect to the individual modules, section protocol data such as section 1 protocol data 280a through section N protocol data 280n dictate the various protocols that govern the individual sections of a given page. In various embodiments, the section protocol data might dictate things such as, but not limited to, the number of sections on a given page, as well as the position, size, and general layout of the sections on the page.

Likewise, each page section may include different types of content, such as text content, image content, video content and user experience (UX) content. For example, in the illustrative embodiment of FIG. 2C, section 1 text content 275a and section N text content 275n are respectfully governed by text content protocol data 285a and text content protocol data 285n. Section 1 image content 277a and section N image content 277n are respectfully governed by image content protocol data 282a and image content protocol data 282n. Section 1 video content 278a and section N video content 278n are respectfully governed by video content protocol data 283a and video content protocol data 283n. Section 1 UX content 279a and section NUX content 279n are respectfully governed by UX content protocol data 284a and UX content protocol data 284n. Text protocol data, image content protocol data, video content protocol data, and UX content protocol data may govern things such as, but not limited to the content of the text, images, videos or UX elements that are present in each section of a given page.

Lastly, protocols can be defined on a very granular level, down to individual words or sequences of words. As illustrated in FIG. 2C, section 1 text content 275a may include a variety of word sequences, such as word sequence 1A (276aa), word sequence 1B (276ab), and word sequence 1C (276ac). Each of these word sequences may be governed by specific protocol data, such as word sequence protocol data 281a. Likewise, section N text content 275n may include a variety of word sequences, such as word sequence NA (276na), word sequence NB (276nb), and word sequence NC (276nc). Each of these word sequences may be governed by specific protocol data, such as word sequence protocol data 281n.

As can be seen from the illustrative embodiments of FIG. 2B and FIG. 2C, each individual therapy can be divided up into any number of sections and sub-sections, each with their own layer of protocols. Thus one therapy could contain thousands of individual protocols and there could be millions of ways of defining, combining, and arranging those protocols to create a protocol or combination of protocols that will be effective for use in administration of a given therapy. In the embodiments disclosed herein, the goal is the generation of improved protocols, or maximally effective protocols, wherein the maximally effective protocols are protocols that are the most effective, during a particular period of time, for the general patient population, the most effective for a particular group of patients, and/or the most effective for a specific individual patient.

Returning now to FIG. 2A, as will now be discussed in detail, the embodiments disclosed herein utilize machine learning models to generate improved and/or maximally effective therapeutic protocols for administration to one or more patients. In order to utilize machine learning models to generate improved and/or maximally effective therapeutic protocols, one or more therapeutic protocol effectiveness prediction models must first be trained, which is depicted in protocol effectiveness prediction training environment 200 of FIG. 2A. Once therapy 212 has been selected for administration to patient 218, historical protocol definition data 206 is retrieved from therapeutic protocol database 202. As used herein, the term “historical therapeutic protocol” may include protocols that have previously been generated, tested, established, and/or clinically validated for use in administration of a therapy. Historical therapeutic protocol data 204 may include data such as historical protocol definition data 206, which defines one or more historical protocols. In the embodiments disclosed herein, it is expected that the number of historically defined protocols will be a very large number, due to the millions of ways of defining and combining those protocols, as noted above. In one embodiment, historical therapeutic protocol data 204 also includes historical protocol effectiveness data 208, which quantifies the effectiveness of each protocol or each combination of protocols represented by historical protocol definition data 206, as will be discussed in additional detail below.

As noted above, in various embodiments, the historical therapeutic protocol data 204 of therapeutic protocol database 202 is specific to a particular culture. For instance, if generation of maximally effective therapeutic protocols for North American patients is the desired goal, then the historical therapeutic protocol data 204 will contain data related to the definition and effectiveness of historical protocols for North American patients. Likewise, if generation of maximally effective therapeutic protocols for Japanese patients is the desired goal, then the historical therapeutic protocol data 204 will contain data related to the definition and effectiveness of historical protocols for Japanese patients.

It should also be noted that, prior to, or during, the training of the therapeutic protocol effectiveness prediction models 260, a health practitioner or other expert in the field may manually select appropriate protocols for inclusion in the therapeutic protocol database 202, and/or may monitor the training process to ensure that invalid, ineffective, and/or inappropriate protocols are not introduced into the therapeutic protocol database 202.

In various embodiments, once the historical protocol definition data 206 is retrieved from therapeutic protocol database 202, a determination is made as to which historical therapeutic protocols 214 to use for administration of therapy 212 to patient 218. In one embodiment, this determination may be made at the discretion of health practitioner 213, who, in some embodiments, is the health practitioner responsible for the treatment of patient 218, and the determination may be based on a wide variety of factors, such as, but not limited to, the severity of the patient's symptoms, the patient's medical history, the patient's age group, the patient's sex, and the patient's ethnicity.

In one embodiment, once the selected therapy 212 is administered to patient 218 according to historical therapeutic protocols 214, the patient's responses to the therapy 212 and associated historical therapeutic protocols 214 are monitored to obtain patient protocol response data 226, which, in some embodiments, may then be stored in a data structure, such as patient database 224. As used herein, in various embodiments, “patient response data” or “patient protocol response data” may include feedback from the patient related to the historical therapeutic protocols 214 utilized in administration of the therapy 212. Patient protocol response data 226 may include direct verbal or written feedback, indirect feedback, such as an indication of whether a particular therapeutic protocol appears to be having an effect on the patient. The patient protocol response data 226 may further include any other measureable data such as, but not limited to, click-stream data showing details related to patient engagement with the content of the therapy, such as, but not limited to, the time that the patient spends engaging with each section of a particular therapy module. The patient protocol response data 226 may also include data received from devices such as, but not limited to, sleep trackers, or other types of physiological sensors that may be used to measure a patient's physiological state, such as heart rate, respiratory rate, and/or blood pressure. The patient protocol response data 226 may then be provided to protocol effectiveness determination module 228, which in one embodiment is responsible for analyzing the patient protocol response data 226 to determine and assign an effectiveness rating to one or more protocols or one or more combinations of protocols.

As one specific illustrative example, one session or module of the therapy 212 might be focused on learning to relax, improving sleep, and managing stress and emotions. A wide variety of data can be collected for classification as patient protocol response data 226. For example, health practitioner 213 may solicit direct feedback from the patient 218 after completion of the therapy session, such as in a verbal interaction with the patient, or through a survey or questionnaire administered in person or remotely, which asks the patient related questions, such as “How would you rate the content of the ‘relaxation’ segment of the module you just completed?”, “Do you feel that you have learned valuable skills from the ‘managing stress’ segment of the module you just completed?”, “Was there anything you didn't like about the ‘improving sleep’ segment of the module you just completed?” A patient may also provide this type of information without solicitation. These types of questions provide one way to quantify the effectiveness of a protocol or a combination of protocols. Indirect response data may also be collected. Continuing the above example, at some point after completion of the above-described module, the patient may be asked questions such as “Do you feel that your stress levels have increased, decreased, or stayed the same since your last session?”, “Has your quality of sleep increased, decreased, or stayed the same since you last session?” These types of questions aren't specifically asking for the patient's direct feedback on a protocol or set of protocols utilized in the therapy, but are instead designed to determine whether the protocols utilized by the therapy have generated the intended result in the patient (e.g. decreased stress and increased quality of sleep would be an indication that the protocols were effective). Additionally, data received from devices such as heart rate monitors, blood pressure monitors, and sleep trackers, may also provide indications as to whether the patient's stress levels have increased or decreased, or whether the patient's quality of sleep has increased or decreased.

In practice, patient feedback, such as patient protocol response data 226, is typically collected in a structured manner using established clinical procedures to ensure the validity of the data interpretation. In various embodiments, the results of the data interpretation may sometimes be referred to as “clinically validated outcome measures,” which may typically be defined as tools that are used in clinical settings to assess the current status of a patient. With respect to the embodiments disclosed herein, analysis of clinically validated outcome measures for a patient can help to determine protocol effectiveness. In various embodiments, an effectiveness rating may be a measure of any number of factors, such as, but not limited to, whether a protocol or a combination of protocols reduces symptom severity, eliminates symptoms, results in improved mood, and/or results in better quality of life for the patient.

In one embodiment, once protocol effectiveness determination module 228 has assigned effectiveness ratings to one or more protocols or combination of protocols, this data may also be stored in a data structure, such as patient database 224, as patient protocol effectiveness data 230, for further use. In one embodiment, the patient protocol effectiveness data 230 generated by protocol effectiveness determination module 228 may also be incorporated into historical protocol effectiveness data 208 of therapeutic protocol database 202.

It should be noted again that the above examples are given for illustrative purposes, and are not intended to limit the scope of the invention as disclosed and claimed herein. One of ordinary skill in the art will readily appreciate that there are many different ways to determine and measure the effectiveness of various protocols and combinations of protocols that are used in administration of a therapy. Application of the historical therapeutic protocols discussed herein typically results in outcome measures that can be clinically validated and thus can reliably be associated with one or more related measures of effectiveness.

As to be expected however, although one particular protocol or combination of protocols may be effective for the general patient population associated with a given culture, or for the average patient associated with a given culture, the same protocol may not be effective at all for a particular patient, or a particular type of patient associated with the given culture. As one simplified example, protocol effectiveness determination module 228 might determine that a protocol utilizing phrase X in a therapy module is only 75% effective when administered in a therapy to patient A, however a protocol utilizing phrase Y in a therapy module is determined to be 90% effective when administered to the same patient A. If the same protocol utilizing phrase X is administered to patient B, it might be found that phrase X is 95% effective for patient B, whereas phrase Y may only be 50% effective for patient B. Thus, it should be clear that the effectiveness ratings of various protocols are likely to vary significantly depending on the characteristics of a particular patient associated with a given culture.

It follows then, that in order to train one or more culture-specific therapeutic protocol effectiveness prediction models, such as therapeutic protocol effectiveness prediction models 260, model training data that accounts for differences in protocol effectiveness among different types of patients within a given culture must be gathered and assembled. In various embodiments, the system and method disclosed herein utilizes patient profile generation module 234 to build a plurality of patient profiles based on data such as patient data 232 of patient database 224. As used herein, the term “patient data” may include data associated with the patient, for example, patient characteristics such as, but not limited to age, sex, ethnicity, religion, marital status, income level, geographic location, personal and family medical history, including the current medical issue that the therapy is designed to treat.

In one embodiment, patient profile generation module 234 may correlate patient protocol effectiveness data 230 with specific profiles in the plurality of generated patient profiles, and, in one embodiment, may store the patient profile data in a data structure, such as patient profile database 236, for later use. Patient profile generation module 234 may generate any number of patient profiles, which may be characterized by the various combinations of patient characteristics represented by patient data 232. As shown in the illustrative embodiment of FIG. 2A, patient profile data 237 of patient profile database 236 includes type 1 patient profile 238, and type 2 patient profile 244 through type n patient profile 250. In one embodiment, type 1 patient profile 238 includes type 1 patient data 240 and type 1 patient protocol effectiveness data 242, type 2 patient profile 244 includes type 2 patient data 246 and type 2 patient protocol effectiveness data 248, and type n patient profile 250 includes type n patient data 252 and type n patient protocol effectiveness data 254, where n can represent any number of patient profiles, depending on the number of patient groupings within the given culture that a user of the method and system disclosed herein wishes to create.

Referring briefly now to FIG. 1 and FIG. 2A together, if the first culture represented by culture 1 historical therapeutic protocol data 107 is North American culture, then culture 1 protocol effectiveness prediction training environment 102 would be utilized to generate trained therapeutic protocol effectiveness prediction models for North American patients, such as trained culture 1 therapeutic protocol effectiveness prediction models 104. Thus, patient profile database 236 of protocol effectiveness prediction training environment 200 would contain patient profile data specific to North American patients.

Returning now to FIG. 2A, as specific illustrative examples, type 1 patient data 240 of type 1 patient profile 238 may describe a patient who is a male, between the ages of 10 and 15, living on the west coast of the United States, who has been diagnosed with irritable bowel syndrome (IBS). Type 2 patient data 246 of type 2 patient profile 244 may describe a patient who is a female, between the ages of 55 and 60, living on the east coast of the United States, who has been diagnosed with breast cancer. Type n patient data 252 of type n patient profile 250 may describe a patient who is a male, between the ages of 65 and 70, living in the southeastern part of the United States, who has been diagnosed with post-traumatic stress disorder (PTSD). In some embodiments, a patient profile of patient profile data 237 may describe a specific patient instead of a group of patients.

In various embodiments, the patient protocol effectiveness data, such as type 1 patient protocol effectiveness data 242, would represent a measure of how effective particular protocols are for patients of that particular type within the given culture. In one embodiment, patient protocol effectiveness data may include a list of hundreds, thousands, or millions of protocols and combinations of protocols, each with corresponding data indicating an effectiveness rating for each protocol or combination of protocols. In some embodiments, an effectiveness rating for a protocol among a particular type of patient within a given culture may be a single number representing an average of the effectiveness ratings for that protocol across all members of the group of patients defined by the patient profile type. In some embodiments an effectiveness rating for a protocol among a particular type of patient within a given culture may be a range of numbers representing the effectiveness ratings for that protocol across all members of the group of patients defined by the patient profile type. In various other embodiments, a weighting system might be utilized, for instance to give higher weight to effectiveness ratings that are more common than others. For example, a particular protocol might have a wide range of effectiveness values for a particular patient profile type, for example, from 30% to 70% effectiveness, however it may be the case that only one or two patients were associated with the 30% effectiveness rating and only one or two patients were associated with the 70% effectiveness rating, however most patients for that profile type were associated with a 60% rating, and so the 60% rating would receive a higher weight that than the other ratings. It should be noted again here that the above examples are given for illustrative purposes only and are not intended to limit the scope of the invention as disclosed and claimed herein.

In various embodiments, once a plurality of patient profiles are generated and associated with protocol effectiveness data, data correlation module 256 of machine learning training module 255 collects patient profile data 237 from patient profile database 236, and historical therapeutic protocol data 204 from therapeutic protocol database 202 and correlates the data to prepare it for transformation into therapeutic protocol effectiveness model training data 258.

In various embodiments, and largely depending on the machine learning based models used, the patient profile data 237 and/or the historical therapeutic protocol data 204 is processed using various methods known in the machine learning arts to identify elements and to vectorize the patient profile data 237 and/or the historical therapeutic protocol data 204. As a specific illustrative example, in a case where the machine leaning based model is a supervised model, the historical therapeutic protocol data 204 and the patient profile data 237 can be analyzed and processed to identify individual elements found to be indicative of protocol effectiveness among certain types of patients, or among a generalized population of patients. These individual elements are then used to create protocol effectiveness data vectors in multidimensional space, resulting in therapeutic protocol effectiveness model training data 258. Therapeutic protocol effectiveness model training data 258 is then used as input data for training one or more machine learning models, such as therapeutic protocol effectiveness prediction models 260. The protocol effectiveness data for a patient profile that correlates with the protocol effectiveness data vector associated with that patient profile is then used as a label for the resulting vector. In various embodiments, this process is repeated for each protocol defined by historical protocol definition data 206 of therapeutic protocol database 202, and for each patient profile type represented by patient profile data 237 of patient profile database 236. The result is that multiple, often millions, of correlated pairs of protocol effectiveness data vectors and patient profiles (as represented by therapeutic protocol effectiveness model training data 258) are used to train one or more machine learning based models, such as therapeutic protocol effectiveness prediction models 260. Consequently, this process results in the creation of one or more trained therapeutic protocol effectiveness prediction models 262. Those of skill in the art will readily recognize that there are many different types of machine learning based models known in the art, and as such, it should be noted that the specific illustrative example of a supervised machine learning based model discussed above should not be construed as limiting the embodiments set forth herein.

For instance, in various embodiments, the one or more machine learning based models can be one or more of: supervised machine learning-based models; semi supervised machine learning-based models; unsupervised machine learning-based models; classification machine learning-based models; logistical regression machine learning-based models; neural network machine learning-based models; deep learning machine learning-based models; and/or any other machine learning based models discussed herein, known at the time of filing, or as developed/made available after the time of filing.

Referring now to FIG. 1 and FIG. 2A together, the trained therapeutic protocol effectiveness prediction models 262, as generated by protocol effectiveness prediction training environment 200 of FIG. 2, correspond to trained culture 1 therapeutic protocol effectiveness prediction models 104 of FIG. 1, for the first culture represented by culture 1 historical therapeutic protocol data 107. Referring now to FIG. 1, as will be discussed in further detail below, in various embodiments, the therapeutic protocol effectiveness prediction models associated with a given culture, such as trained culture 1 therapeutic protocol effectiveness prediction models 104, can be used in a variety of ways. In one embodiment, the trained therapeutic protocol effectiveness prediction models associated with a given culture can be used to dynamically generate one or more improved or maximally effective therapeutic protocols for a specific patient, or for a specific type of patient associated with the given culture. In one embodiment, in the case of dynamically generating one or more improved or maximally effective therapeutic protocols for a specific patient associated with the given culture, a psychological therapy is selected for administration to the patient. In the illustrative embodiment of FIG. 1, trained therapeutic protocol effectiveness prediction models for a given culture, such as trained culture 1 therapeutic protocol effectiveness prediction models 104, are incorporated into an effective protocol generation runtime environment associated with a given culture, such as culture 1 effective protocol generation runtime environment 106. In one embodiment, the effective protocol generation runtime environment associated with a given culture analyzes patient data associated with the patient and patient profile data associated with predefined patient profiles to select a patient profile that is the best match for the specific patient, and the selected patient profile data is provided to the trained therapeutic protocol effectiveness prediction models associated with the given culture. In one embodiment, new therapeutic protocol test data is generated or otherwise obtained, and is also provided to the trained therapeutic protocol effectiveness prediction models associated with the given culture.

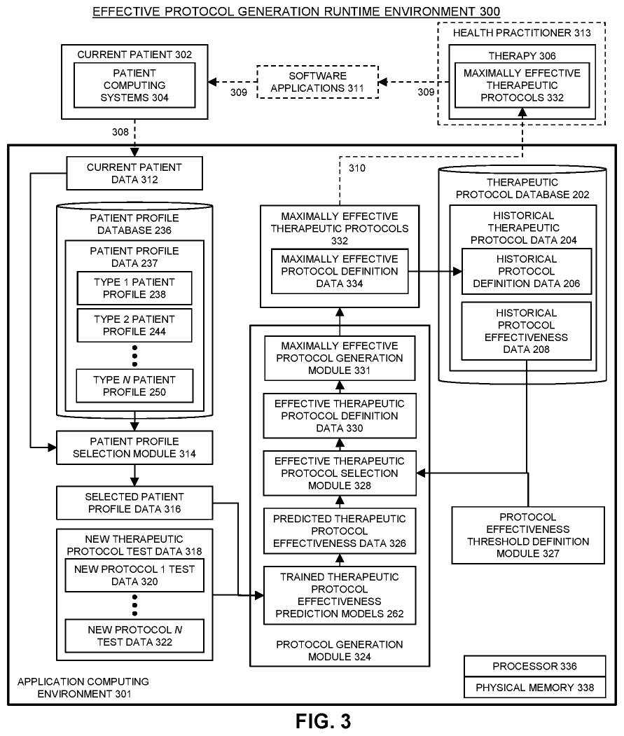

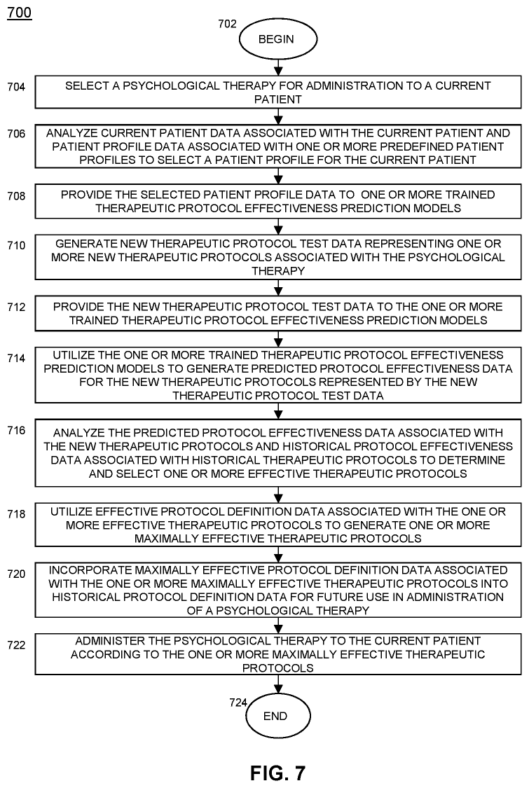

In one embodiment, the trained therapeutic protocol effectiveness prediction models associated with the given culture are utilized to generate predicted protocol effectiveness data for the new protocols associated with the given culture. In one embodiment, predicted protocol effectiveness data, and historical protocol effectiveness data are analyzed to determine and select one or more effective therapeutic protocols, which are utilized to generate one or more maximally effective therapeutic protocols for patients associated with the given culture. In one embodiment, maximally effective protocol definition data associated with the one or more maximally effective therapeutic protocols is stored as historical protocol definition data for future use in administration of a psychological therapy. In one embodiment, the selected psychological therapy is then administered to the patient according to the maximally effective therapeutic protocols. The above described system and process will be discussed in additional detail below with reference to the system of FIG. 3 and the process of FIG. 7.

FIG. 3 is a block diagram of an effective protocol generation runtime environment 300 for utilizing trained therapeutic protocol effectiveness prediction models to generate maximally effective therapeutic protocols for a specific patient, in accordance with one embodiment.

In various embodiments, effective protocol generation runtime environment 300 includes application computing environment 301, current patient 302 and associated patient computing systems 304, software applications 311, health practitioner 313, therapy 306 and associated maximally effective therapeutic protocols 332. In one embodiment, maximally effective therapeutic protocols 332 include maximally effective protocol definition data 334. In one embodiment, effective protocol generation runtime environment 300 further includes communications channel 308, which facilitates transmission of data from current patient 302 to application computing environment 301, communications channel 309, which facilitates administration of therapy 306 to current patient 302, and communications channel 310, which facilitates retrieval of data from application computing environment 301 to be incorporated in therapy 306. Each of the above listed elements will be discussed in further detail below.

In various embodiments, application computing environment 301 includes therapeutic protocol database 202, and patient profile database 236. In one embodiment, therapeutic protocol database 202 includes historical therapeutic protocol data 204, which further includes historical protocol definition data 206, and historical protocol effectiveness data 208. In one embodiment, patient profile database 236 includes patient profile data 237, which further includes type 1 patient profile 238, and type 2 patient profile 244 through type n patient profile 250. Each of the above listed elements will be discussed in further detail below.

In various embodiments, application computing environment 301 further includes additional data such as current patient data 312, selected patient profile data 316, and new therapeutic protocol test data 318, which further includes new protocol 1 test data 320 through new protocol n test data 322. In one embodiment, application computing environment 301 further includes several process modules, such as patient profile selection module 314, protocol generation module 324, and protocol effectiveness threshold definition module 327. In various embodiments, protocol generation module 324 includes trained therapeutic protocol effectiveness prediction models 262, predicted therapeutic protocol effectiveness data 326, effective therapeutic protocol selection module 328, effective therapeutic protocol definition data 330, and maximally effective protocol generation module 331. In one embodiment, application computing environment 301 further includes processor 336 and physical memory 338, which together coordinate the operation and interaction of the data and data processing modules associated with application computing environment 301. Each of the above listed elements will be discussed in further detail below.

In one embodiment, current patient 302 is a patient associated with a given culture, who has been diagnosed with a medical condition, and a determination is made as to whether current patient 302 will benefit from receiving one or more therapies. As noted above, there are a variety of established and/or clinically validated therapies that have been shown to provide benefit to patients, and administration of these clinically validated therapies are typically governed by a collection of therapeutic protocols associated with the particular therapy. In one embodiment, once a determination has been made that current patient 302 is likely to benefit from a particular therapy, such as therapy 306, the previously trained therapeutic protocol effectiveness prediction models 262 associated with the given culture are utilized to generate one or more protocols that will be maximally effective for current patient 302.

In order to achieve this outcome, in one embodiment, current patient data 312 is obtained, either directly from current patient 302 and/or patient computing systems 304, from medical files associated with current patient 302 and/or current patient data 312 may be retrieved from a database of previously collected patient data. As noted above, and as used herein, the term “patient data” may include data associated with the patient, for example, patient characteristics such as, but not limited to age, sex, ethnicity, religion, marital status, income level, geographic location, personal and family medical history, including the current medical issue that the therapy is designed to treat. In various embodiments, patient computing systems 304 may include, but are not limited to, a desktop computing system, a mobile computing system, a virtual reality computing system, a gaming computing system, a computing system that utilizes one or more Internet of Things (IoT) devices, or any combination thereof.

In some embodiments, there may be no specific current patient 302, and test data may be used in place of current patient data 312. For example, a theoretical patient may be contemplated, and data describing characteristics of the theoretical patient may be used as test data in place of current patient data 312 to generate maximally effective therapeutic protocols for the theoretical patient. In various embodiments, test data may be generated by one or more machine learning models that have been trained to predict effectiveness of the test data.

In various embodiments, once the current patient data 312 has been obtained for current patient 302, patient profile selection module 314 analyzes the current patient data 312 along with the patient profile data 237 of patient profile database 236, in order to select a patient profile that most closely matches the characteristics of current patient 302. In one embodiment, patient profile data 237 contains profile data for patients associated with the same culture as current patient 302.

In one embodiment, patient profile selection module 314 compares various characteristics of current patient 302 to patient characteristics represented by the one or more patient profiles in the patient profile database 236. As will be noted by those of skill in the art, various mechanisms and algorithms may be utilized to determine similarities between current patient 302 and the patient profiles represented by patient profile data 237. Similarity of current patient 302 to a particular patient profile may be determined by any number of factors, such as, but not limited to current patient 302's age, sex, ethnicity, religion, marital status, income level, geographic location, personal and family medical history, including the current medical issue that the therapy is designed to treat.

In one embodiment, one or more thresholds may be defined to determine how close of a match current patient 302 is to a particular patient profile. For example, if current patient 302's characteristics match with type 1 patient profile 238 by 60%, but match with type 2 patient profile 244 by 80%, and no other patient profile type is found to provide a better match, the patient profile with the closest match may be selected for utilization in determination of an effective therapeutic protocol to utilize for treating current patient 302.

Continuing the specific illustrative examples given above, current patient 302 may be a male, age 13, living in California, who has been diagnosed with IBS, and so may be classified as a ‘type 1’ patient, wherein the ‘type 1’ patient is associated with type 1 patient profile 238, which describes a patient associated with North American culture, who is a male, between the ages of 10 and 15, living on the west coast of the United States, who has been diagnosed with IBS, and so patient profile selection module 314 may determine that current patient 302 should be associated with type 1 patient profile 238, and this selection may be represented by selected patient profile data 316. It should be noted herein that the above examples are simplified, and are given for illustrative purposes only. One of skill in the art will readily recognize that the millions of different combinations of patient characteristics, the models that govern the interactions between those characteristics, and the protocols associated with the treatment administered to those patients, requires a vast amount of data collection and analysis which simply cannot be performed by the human mind alone, even with the aid of pen and paper and even given unlimited time to accomplish the task.

In various embodiments, once patient profile selection module 314 has determined selected patient profile data 316, the selected patient profile data 316 is provided as input to the one or more trained therapeutic protocol effectiveness prediction models 262, along with new therapeutic protocol test data 318. As discussed above, in various embodiments, and as used herein, the terms “current therapeutic protocol” or “historical therapeutic protocol” refer to a protocol that has previously been generated, tested, established, and/or clinically validated for use in administration of a therapy. Likewise, in one embodiment, the terms “new protocol,” and “new therapeutic protocol” refer to a protocol that has not been previously generated, tested, established, and/or clinically validated for use in administration of a therapy. Additionally, in some embodiments, the terms “new protocol,” and “new therapeutic protocol” may refer to a protocol that has been previously generated and/or tested, but may not yet be established and/or clinically validated for use in administration of a therapy. In various embodiments, new therapeutic protocols may also be thought of as potential therapeutic protocols or candidate therapeutic protocols, in the sense that they are protocols that are being considered for use in a therapy.

As one simplified example, a historical therapeutic protocol for a cognitive behavioral therapy may dictate that the therapy should contain eight modules that are presented to the patient in a particular order, such as 1, 2, 3, 4, 5, 6, 7, 8. A new therapeutic protocol might dictate that there should be nine modules, presented to the patient in a different order, such as 1, 2, 4, 3, 6, 5, 8, 7, 9. Similarly, a historical therapeutic protocol for a cognitive behavioral therapy may dictate that a page section of module 6 should include text content that contains the word sequence “alternatives to negative thoughts,” whereas a new therapeutic protocol may dictate that the same page section of module 6 should instead include text content that contains the word sequence “alternatives to unhelpful thoughts.”

In one embodiment, new therapeutic protocol test data 318 of FIG. 3 includes data representing any number of new therapeutic protocols, such as new protocol 1 through new protocol n, which are represented by new protocol 1 test data 320 through new protocol n test data 322. In one embodiment, once the selected patient profile data 316 and new therapeutic protocol test data 318 have been provided as input to the one or more trained therapeutic protocol effectiveness prediction models 262 of protocol generation module 324, the one or more trained therapeutic protocol effectiveness prediction models 262 generate predicted therapeutic protocol effectiveness data 326. In various embodiments, predicted therapeutic protocol effectiveness data 326 represents the predicted effectiveness of each of the new therapeutic protocols represented by new therapeutic protocol test data 318 for a patient who matches the patient profile type represented by selected patient profile data 316, such as current patient 302.

As one simplified example, patient profile selection module 314 might categorize current patient 302 as a match for a type 1 patient, as represented by type 1 patient profile 238 of patient profile data 237. Predicted therapeutic protocol effectiveness data 326 might indicate that a first new protocol is 75% effective for type 1 patients, and a second new protocol is 50% effective for type 1 patients. Likewise, for a patient who has been categorized as a type 2 patient, predicted therapeutic protocol effectiveness data 326 might indicate that the same first new protocol is 30% effective for type 2 patients, and the same second new protocol is 90% effective for type 2 patients. Thus, the output of the trained therapeutic protocol effectiveness prediction models 262, predicted therapeutic protocol effectiveness data 326, is dependent on both the new therapeutic protocol test data 318, as well as the patient profile type represented by selected patient profile data 316.

As discussed above, effectiveness of a protocol or a combination of protocols can be determined and defined in a number of ways, including, but not limited to, analysis of direct or indirect feedback from a patient, analysis of patient physiological data, and/or analysis of a variety of clinically validated outcome measures. An effectiveness rating may be a measure of any number of factors, such as, but not limited to, whether a protocol or a combination of protocols reduces symptom severity, eliminates symptoms, results in improved mood, and/or results in better quality of life for the patient.

In one embodiment, once predicted therapeutic protocol effectiveness data 326 has been generated by trained therapeutic protocol effectiveness prediction models 262, it is passed to effective therapeutic protocol selection module 328 of protocol generation module 324 for further analysis. In one embodiment, effective therapeutic protocol selection module 328 selects one or more of the new therapeutic protocols represented by new therapeutic protocol test data 318 that have been found to be effective. A determination as to what constitutes an “effective” protocol may be made in any number of ways. As one illustrative example, protocol effectiveness threshold definition module 327 may set one or more threshold values for the effectiveness ratings represented by predicted therapeutic protocol effectiveness data 326. In one embodiment, protocol effectiveness threshold definition module 327 may be separate from protocol generation module 324. In one embodiment, protocol effectiveness threshold definition module 327 may be a sub-module of protocol generation module 324. In one embodiment, one or more threshold values may be explicitly set, for example, based on input from one or more health practitioners. In various other embodiments, protocol effectiveness threshold definition module 327 may derive or learn one or more threshold values based on analysis of training data, such as, but not limited to historical protocol effectiveness data 208. As one simplified example, in one embodiment, protocol effectiveness threshold definition module 327 may define an effectiveness threshold such that any protocol having a known or predicted effectiveness rating of 75% or higher should be considered an “effective” protocol by effective therapeutic protocol selection module 328. In one embodiment, effective therapeutic protocol selection module 328 may also consider historical protocol effectiveness data 208 in determining and selecting effective protocols.

Continuing the above simplified example, a historical therapeutic protocol for a cognitive behavioral therapy may dictate that the therapy should contain eight modules that are presented to the patient in a particular order, such as 1, 2, 3, 4, 5, 6, 7, 8. A new therapeutic protocol might dictate that there should be nine modules, presented to the patient in a different order, such as 1, 2, 4, 3, 6, 5, 8, 7, 9. It may be found that the historical protocol has a known effectiveness rating of 90%, whereas the new therapeutic protocol has a predicted effectiveness rating of 80%. In the illustrative embodiment where protocol effectiveness threshold definition module 327 has set the threshold value for effectiveness ratings to 75%, the effective therapeutic protocol selection module 328 may determine that, while the new protocol is predicted to be effective, the historical protocol is actually known to be more effective, and so the historical protocol may be selected over the new protocol. Similarly, a historical therapeutic protocol for a cognitive behavioral therapy may dictate that a page section of module 6 should include text content that contains the word sequence “alternatives to negative thoughts,” whereas a new therapeutic protocol may dictate that the same page section of module 6 should instead include text content that contains the word sequence “alternatives to unhelpful thoughts.” The historical protocol may have a known 75% effectiveness rating, but the new protocol may have a predicted 85% rating, and so effective therapeutic protocol selection module 328 may select the new protocol.

In one embodiment, once effective therapeutic protocol selection module 328 has selected one or more effective therapeutic protocols, effective therapeutic protocol definition data 330 is generated, which contains data defining the one or more selected effective protocols. In one embodiment, maximally effective protocol generation module 331 utilizes effective therapeutic protocol definition data 330 to generate one or more maximally effective therapeutic protocols 332. As used herein, the term “maximally effective protocol” or “maximally effective therapeutic protocol” may include therapeutic protocols that have been determined to be the most effective therapeutic protocols, for a particular period of time, out of the new, current, and/or historical effective therapeutic protocols. In various embodiments, maximally effective therapeutic protocols 332 may include any number and combination of maximally effective protocols, and each of these protocols or protocol combinations is defined by maximally effective protocol definition data 334. Continuing the above illustrative example, protocol generation module 324 may determine that using the word sequence “alternative to unhelpful thoughts” in place of “alternative to negative thoughts” is maximally effective for the patient profile type represented by selected patient profile data 316, independently of the other protocols in the therapy. Protocol generation module 324 may instead determine that using the word sequence “alternative to unhelpful thoughts” in place of “alternative to negative thoughts” is only effective when presented on page one of module six of a therapy that has eight modules.

It should be noted here that the above simplified examples are given for illustrative purposes only and are not intended to limit the invention as disclosed and claimed herein. It should be readily apparent to those of ordinary skill in the art that there are millions of potential protocols and protocol combinations that may be employed in a therapy, and so the generation of maximally effective protocols and protocol combinations is not a task that can be accomplished in the human mind, even with pen and paper, and even given unlimited time.

Referring briefly to FIG. 2A and FIG. 3 together, in various embodiments, once one or more maximally effective therapeutic protocols 332 have been generated by maximally effective protocol generation module 331 of protocol generation module 324, the maximally effective protocol definition data 334 representing the maximally effective therapeutic protocols 332 may be stored in a data structure, such as therapeutic protocol database 202, for further use. For example, in one embodiment, maximally effective protocol definition data 334 is incorporated into historical protocol definition data 206 of historical therapeutic protocol data 204. This is advantageous because it creates a feedback loop for the machine learning process, wherein the newly generated maximally effective therapeutic protocols 332 can be incorporated into the therapeutic protocol effectiveness model training data 258 of FIG. 2A, which is used to train the therapeutic protocol effectiveness prediction models 260. In this manner, the trained therapeutic protocol effectiveness prediction models 262 may be continually updated and refined as new patient protocol response data 226 is received from patient 218.

Returning now to FIG. 3, in one embodiment, once one or more maximally effective therapeutic protocols 332 have been generated by maximally effective protocol generation module 331 of protocol generation module 324, the one or more maximally effective therapeutic protocols 332 may be incorporated into a therapy, such as therapy 306, which may then be administered to a patient, such as current patient 302. In some embodiments, once generated, the maximally effective therapeutic protocols 332 may be automatically incorporated into a therapy, such as therapy 306, for administration to current patient 302. In some embodiments, a health practitioner, such as health practitioner 313, may review maximally effective therapeutic protocols 332 prior to incorporation into therapy 306 for administration to current patient 302. In some embodiments, the maximally effective therapeutic protocols 332 may be stored in a data structure, such as therapeutic protocol database 202, for further use, but might not be incorporated into a particular therapy. In some embodiments, the one or more maximally effective therapeutic protocols 332 may be incorporated into a therapy, such as therapy 306, but the therapy 306 may not be administered to current patient 302 and/or the therapy 306 may be administered to a patient other than current patient 302.

In various embodiments, the therapy 306 may be administered to current patient 302 using one or more communication mechanisms 309. In some embodiments, communication mechanisms 309 include health practitioner 313 conducting a physical in-person meeting with current patient 302 to verbally guide current patient 302 through the therapy 306. In other embodiments, communication mechanisms 309 include administering the therapy 306 to current patient 302 remotely, for example through a website, or through one or more software applications 311 that can be executed from patient computing systems 304. In one embodiment, the therapy 306 may be administered to current patient 302 directly by health practitioner 313. In one embodiment, therapy 306 may be administered to current patient 302 remotely, without the direct involvement of health practitioner 313. For example, therapy 306 may be self-administered by current patient 302. In one embodiment, therapy 306 may also be administered to current patient 302 remotely with partial involvement of health practitioner 313.

In various embodiments, patient computing systems 304 may include, but are not limited to, a desktop computing system, a mobile computing system, a virtual reality computing system, a gaming computing system, a computing system that utilizes one or more Internet of Things (IoT) devices, or any combination thereof.

In one embodiment, regardless of whether the one or more maximally effective therapeutic protocols 332 are incorporated into therapy 306, and/or administered to current patient 302, the protocols that were predicted to be maximally effective for patients associated with a first culture may be further utilized to facilitate rapid generation of protocols that will be maximally effective for one or more cultures other than the first culture, as will be discussed in further detail below.

As will be discussed in further detail below, in addition to the embodiments discussed above, trained therapeutic protocol effectiveness prediction models that are associated with a given culture can be utilized independently of a specific patient or specific type of patient, for example, to generate one or more improved or maximally effective therapeutic protocols that are generally effective for patients associated with the given culture, regardless of the other non-cultural aspects of the patients' background and history. In one embodiment, in the case of generating improved or maximally effective therapeutic protocols that are generally effective for patients associated with the given culture, or are effective for an average patient associated with the given culture, a system similar to that described above may be utilized, without providing patient-specific profile data to the trained therapeutic protocol effectiveness prediction models.

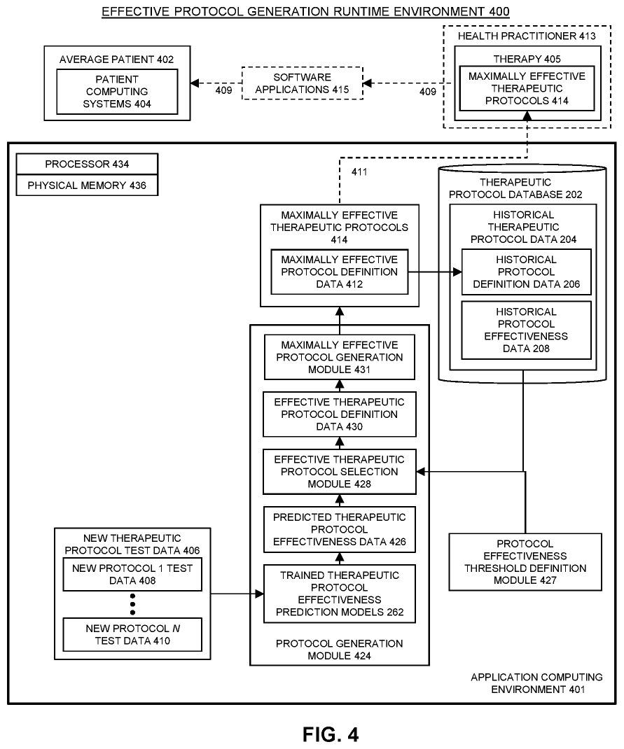

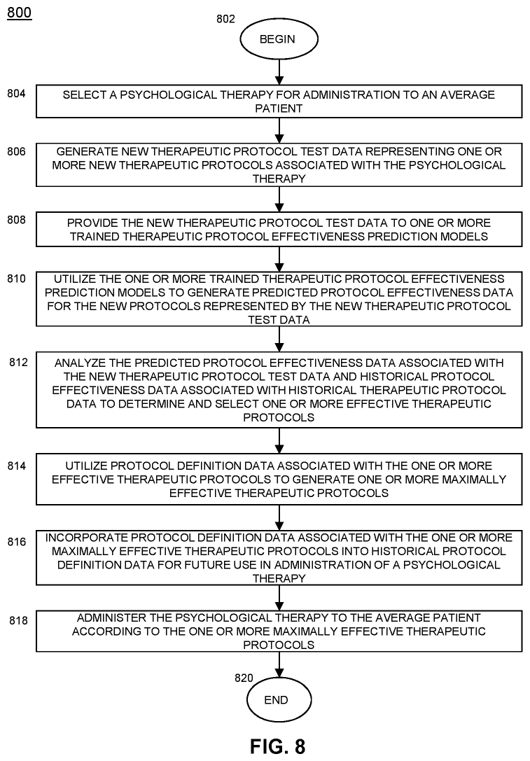

For example, in some embodiments, a psychological therapy is selected for administration to one or more patients, new therapeutic protocol test data is generated and provided to the trained therapeutic protocol effectiveness prediction models associated with the given culture, and the trained therapeutic protocol effectiveness prediction models are utilized to generate predicted protocol effectiveness data for the new protocols associated with the given culture. In some embodiments, the predicted protocol effectiveness data and the historical protocol effectiveness data are analyzed to select one or more effective therapeutic protocols. Protocol definition data associated with the one or more effective therapeutic protocols is then utilized to generate one or more maximally effective therapeutic protocols for patients associated with the given culture. In one embodiment, maximally effective protocol definition data associated with the one or more maximally effective therapeutic protocols is incorporated into historical protocol definition data for future use in administration of the selected psychological therapy. The above described system and process will be discussed in additional detail below with reference to the system of FIG. 4 and the process of FIG. 8.

FIG. 4 is a block diagram of an effective protocol generation runtime environment 400 for utilizing trained therapeutic protocol effectiveness prediction models to generate generalized maximally effective therapeutic protocols, in accordance with one embodiment.

In various embodiments, effective protocol generation runtime environment 400 includes application computing environment 401, average patient 402 and associated patient computing systems 404, software applications 415, health practitioner 413, therapy 405, and maximally effective therapeutic protocols 414. In one embodiment, effective protocol generation runtime environment 400 further includes communications channel 409, which facilitates administration of therapy 405 to average patient 402, and communications channel 411, which facilitates retrieval of data from application computing environment 401. In one embodiment, application computing environment 401 includes therapeutic protocol database 202, which further includes historical therapeutic protocol data 204, such as historical protocol definition data 206, and historical protocol effectiveness data 208. In various embodiments, application computing environment 401 further includes additional data such as new therapeutic protocol test data 406, which further includes new protocol 1 test data 408 through new protocol n test data 410. In one embodiment, application computing environment 401 further includes protocol generation module 424, and protocol effectiveness threshold definition module 427. In various embodiments, protocol generation module 424 includes trained therapeutic protocol effectiveness prediction models 262, predicted therapeutic protocol effectiveness data 426, effective therapeutic protocol selection module 428, effective therapeutic protocol definition data 430, and maximally effective protocol generation module 431. In one embodiment, application computing environment 401 further includes processor 434 and physical memory 436, which together coordinate the operation and interaction of the data and data processing modules associated with application computing environment 401. Each of the above listed elements will be discussed in further detail below.

As noted above, there are a variety of established and/or clinically validated therapies that have been shown to provide benefit to patients, and administration of these clinically validated therapies are typically governed by a collection of therapeutic protocols associated with the particular therapy. In one embodiment, a therapy, such as therapy 405, is selected for administration to one or more patients associated with a given culture, and the previously trained therapeutic protocol effectiveness prediction models 262 associated with the given culture are utilized to generate one or more protocols that will be maximally effective for patients in general or for average patients associated with the given culture.

In one embodiment, new therapeutic protocol test data 406 is generated or otherwise obtained, and is provided as input to the one or more trained therapeutic protocol effectiveness prediction models 262 of protocol generation module 424. In one embodiment, new therapeutic protocol test data 406 of FIG. 4 includes data representing any number of new therapeutic protocols, such as new protocol 1 through new protocol n, which are represented by new protocol 1 test data 408 through new protocol n test data 410. In one embodiment, once the new therapeutic protocol test data 406 has been provided as input to the one or more trained therapeutic protocol effectiveness prediction models 262 of protocol generation module 424, the one or more trained therapeutic protocol effectiveness prediction models 262 generate predicted therapeutic protocol effectiveness data 426. In various embodiments, predicted therapeutic protocol effectiveness data 426 represents the predicted effectiveness of each of the new therapeutic protocols represented by new therapeutic protocol test data 406 for an average patient associated with the given culture, such as average patient 402.

In one embodiment, once predicted therapeutic protocol effectiveness data 426 has been generated by trained therapeutic protocol effectiveness prediction models 262, it is passed to effective therapeutic protocol selection module 428 of protocol generation module 424 for further analysis. In one embodiment, effective therapeutic protocol selection module 428 selects one or more of the new therapeutic protocols represented by new therapeutic protocol test data 406 that have been found to be effective. As discussed above, a determination as to what constitutes an “effective” protocol may be made in any number of ways. As one illustrative example, protocol effectiveness threshold definition module 427 may set one or more threshold values for the effectiveness ratings represented by predicted therapeutic protocol effectiveness data 426. In one embodiment, protocol effectiveness threshold definition module 427 may be separate from protocol generation module 424. In one embodiment, protocol effectiveness threshold definition module 427 may be a sub-module of protocol generation module 424. In one embodiment, one or more threshold values may be explicitly set, for example, based on input from one or more health practitioners. In various other embodiments, protocol effectiveness threshold definition module 427 may derive or learn one or more threshold values based on analysis of training data, including, but not limited to historical protocol effectiveness data 208. In one embodiment, effective therapeutic protocol selection module 428 may also consider historical protocol effectiveness data 208 in determining and selecting effective protocols.

In one embodiment, once effective therapeutic protocol selection module 428 has selected one or more effective therapeutic protocols, effective therapeutic protocol definition data 430 is generated, which contains data defining the one or more selected effective protocols. In one embodiment, maximally effective protocol generation module 431 utilizes effective therapeutic protocol definition data 430 to generate one or more maximally effective therapeutic protocols 414. In various embodiments, maximally effective therapeutic protocols 414 may include any number and combination of maximally effective protocols, and each of these protocols or protocol combinations is defined by maximally effective protocol definition data 412.

Referring briefly to FIG. 2A and FIG. 4 together, in various embodiments, once one or more maximally effective therapeutic protocols 414 have been generated by maximally effective protocol generation module 431 of protocol generation module 424, the maximally effective protocol definition data 412 representing the maximally effective therapeutic protocols 414 may be stored in a data structure, such as therapeutic protocol database 202, for further use. For example, in one embodiment, maximally effective protocol definition data 412 is incorporated into historical protocol definition data 206 of historical therapeutic protocol data 204. As noted above, this is advantageous because it creates a feedback loop for the machine learning process, wherein the newly generated maximally effective therapeutic protocols 414 can be incorporated into the therapeutic protocol effectiveness model training data 258 of FIG. 2A, which is used to train the therapeutic protocol effectiveness prediction models 260. In this manner, the trained therapeutic protocol effectiveness prediction models 262 may be continually updated and refined as new patient protocol response data 226 is received from patient 218.

Returning now to FIG. 4, in one embodiment, once one or more maximally effective therapeutic protocols 414 have been generated by maximally effective protocol generation module 431 of protocol generation module 424, the one or more maximally effective therapeutic protocols 414 may be incorporated into a therapy, such as therapy 405. In some embodiments, the maximally effective therapeutic protocols 414 may be stored in a data structure such as therapeutic protocol database 202, for further use, but might not be incorporated into a particular therapy.

In one embodiment, once maximally effective therapeutic protocols 414 have been incorporated into a therapy, such as therapy 405, therapy 405 may then be administered to a patient, such as average patient 402. In some embodiments, once generated, the maximally effective therapeutic protocols may be automatically incorporated into therapy 405 for administration to average patient 402. In some embodiments, a health practitioner, such as health practitioner 413, may review maximally effective therapeutic protocols 414 prior to incorporation into therapy 405 for administration to average patient 402. In some embodiments, the maximally effective therapeutic protocols 414 may be stored in a data structure, such as therapeutic protocol database 202, for further use, but might not be administered to average patient 402.

In various embodiments, the maximally effective therapeutic protocols 414 may be administered to average patient 402 using one or more communication mechanisms 409. In some embodiments, communication mechanisms 409 include health practitioner 413 conducting a physical in-person meeting with average patient 402 to verbally guide average patient 402 through the therapy 405. In other embodiments, communication mechanisms 409 include administering the therapy 405 to average patient 402 remotely, for example through a website, or through one or more software applications 415 that can be executed from patient computing systems 404. In one embodiment, the therapy 405 may be administered to average patient 402 directly by health practitioner 413. In one embodiment, therapy 405 may be administered to average patient 402 remotely, without the direct involvement of health practitioner 413. For example, therapy 405 may be self-administered by average patient 402. In one embodiment, therapy 405 may also be administered to average patient 402 remotely with partial involvement of health practitioner 413.

In various embodiments, patient computing systems 404 may include, but are not limited to, a desktop computing system, a mobile computing system, a virtual reality computing system, a gaming computing system, a computing system that utilizes one or more Internet of Things (IoT) devices, or any combination thereof.

In one embodiment, regardless of whether the one or more maximally effective therapeutic protocols 414 are incorporated into therapy 405, and/or administered to average patient 402, the protocols that were found to be maximally effective for a first culture may be further utilized to facilitate rapid generation of protocols that will be maximally effective for one or more cultures other than the first culture, as will be discussed in further detail below.

Returning now to FIG. 1, as mentioned above, once maximally effective therapeutic protocols have been generated for a first culture, the maximally effective therapeutic protocols for the first culture may be further utilized to facilitate rapid generation of protocols that will be maximally effective for one or more cultures other than the first culture.

In the illustrative embodiment of FIG. 1, a protocol generation system, such as culture 1 protocol generation system 101, takes in culture 1 historical therapeutic protocol data 107 and outputs culture 1 maximally effective therapeutic protocols 108. As noted above, in some embodiments, a large amount of data regarding protocol effectiveness for patients associated with the first culture may already exist, while there may be very little data available regarding protocol effectiveness for patients associated with cultures other than the first culture. Thus, absent the method and system disclosed herein, the process of generating maximally effective therapeutic protocols for patients associated with cultures other than the first culture may take a great deal more time than generating maximally effective therapeutic protocols for patients associated with the first culture.

In one embodiment, in order to facilitate rapid generation of protocols that will be maximally effective for other cultures, culture 1 maximally effective therapeutic protocols 108 are first provided to protocol translation module 110, which, in some embodiments, is responsible for translating culture 1 maximally effective therapeutic protocols 108 from the language and/or dialect associated with the first culture, to the language and/or dialect of one or more cultures other than the first culture. In the illustrative embodiment of FIG. 1, protocol translation module 110 generates translated protocol data 112, which may include culture 2 translated protocols 114 through culture n translated protocols 116.

In one embodiment, once translated protocol data 112 is generated, it is provided to culturally sensitive protocol translation module 117 to determine whether further modifications are needed. Typically, when translating languages and/or dialects from one culture to another, a literal translation may be generated, and/or the translation may take into account grammatical differences between languages and adjust the translation as appropriate. However, there are many additional culturally-based nuances that should be taken into account when performing a translation, especially in the fields of mental and physical healthcare, where the quality of care that a patient receives may be very much dependent on an understanding of the cultural sensitivities particular to the patient's culture. For example, there are words and phrases that may be commonly used in North American culture that may be confusing, humorous or offensive in many Eastern cultures. While a particular word or phrase might have one connotation in one culture, it may have a completely different connotation in other cultures. In one culture it might be appropriate to ask a particular question, while in other cultures the same question might be considered overly direct, rude or offensive. Additionally, although the words and phrases used in a protocol, as part of a therapy, are very important, other protocol elements may also be important. For example, images, animations, and/or videos that are acceptable in one culture may not be acceptable in another culture. Audio, such as music and sound effects that are preferable in one culture may not be preferable in another culture. Thus, a particular protocol or combination of protocols that may be maximally effective in one culture may not be at all effective in other cultures.

In order to address these issues, data related to the sensitivities of various cultures needs to be taken into account when translating a protocol from the language/dialect of one culture to the language/dialect of another culture. In the illustrative embodiment of FIG. 1, this data is represented by culture 2 sensitivity data 120 through culture n sensitivity data 122, which in one embodiment is stored in a data structure, such as cultural sensitivity database 118. In various embodiments, the cultural sensitivity data may be obtained by any available sources of cultural sensitivity data. In some embodiments, the cultural sensitivity data is automatically obtained from existing repositories of cultural data. In other embodiments, the cultural sensitivity data may be manually generated by one or more linguistic experts in a given culture or cultures.

In the illustrative embodiment of FIG. 1, once protocol translation module 110 has performed an initial translation to generate translated protocol data 112, culture 2 translated protocols 114 through culture n translated protocols 116 are checked against culture 2 sensitivity data 120 through culture n sensitivity data 122 of cultural sensitivity database 118, in order to identify any potential conflicts between the translated protocol data and the traditions, customs, and values that are held by various other cultures.

In various embodiments, in the case of no conflicts between translated protocol data 112 and the traditions, customs, and values held by a particular culture, it may be that no modifications are made to the initial translated protocol data 112, however, if one or more conflicts are identified, then culturally sensitive protocol translation module 117 may modify one or more of culture 2 translated protocols 114 through culture n translated protocols 116, based on culture 2 sensitivity data 120 through culture n sensitivity data 122 to resolve any identified conflicts. In one embodiment, this results in the generation of culturally sensitive translated protocol data 124, which in some embodiments includes culture 2 culturally sensitive translated protocols 126 through culture n culturally sensitive translated protocols 128.

In one embodiment, once translated protocol data 112 has been transformed into culturally sensitive translated protocol data 124 by culturally sensitive protocol translation module 117, the culturally sensitive translated protocol data 124 is provided as input to one or more additional protocol generation systems, such as culture 2 protocol generation system 130 through culture n protocol generation system 140. It should be noted that culture 2 protocol generation system 130 through culture n protocol generation system 140 function in the same manner as culture 1 protocol generation system 101, discussed in detail above, with the only difference being the culture-specific data that is being fed as inputs to the protocol generation system. For example, culture 1 protocol generation system 101 takes culture 1 historical therapeutic protocol data 107 as inputs, culture 2 protocol generation system 130 takes culture 2 culturally sensitive translated protocols 126 as inputs, and culture n protocol generation system 140 takes culture n culturally sensitive translated protocols 128 as inputs.

Similarly to the functioning of culture 1 protocol generation system, culture 2 protocol generation system 130 utilizes culture 2 culturally sensitive translated protocol data 126 to train one or more therapeutic protocol effectiveness prediction models within culture 2 protocol effectiveness training environment 132, resulting in trained culture 2 therapeutic protocol effectiveness prediction models 134. Trained culture 2 therapeutic protocol effectiveness prediction models 134 are then incorporated into culture 2 effective protocol generation runtime environment 136, which generates culture 2 maximally effective therapeutic protocols 138, wherein culture 2 maximally effective therapeutic protocols 138 are protocols that have been determined by culture 2 protocol generation system 130 to be maximally effective for patients associated with culture 2.

Likewise, culture n protocol generation system 140 utilizes culture n culturally sensitive translated protocol data 128 to train one or more therapeutic protocol effectiveness prediction models within culture n protocol effectiveness training environment 142, resulting in trained culture n therapeutic protocol effectiveness prediction models 144. Trained culture n therapeutic protocol effectiveness prediction models 134 are then incorporated into culture n effective protocol generation runtime environment 146, which generates culture n maximally effective therapeutic protocols 148, wherein culture n maximally effective therapeutic protocols 148 are protocols that have been determined by culture n protocol generation system 140 to be maximally effective for patients associated with culture n.

Thus, the above described system is capable of utilizing historical therapeutic protocol data associated with a first culture to dynamically, efficiently, and rapidly generate culturally sensitive therapeutic protocols for any number of different cultures.

### Process

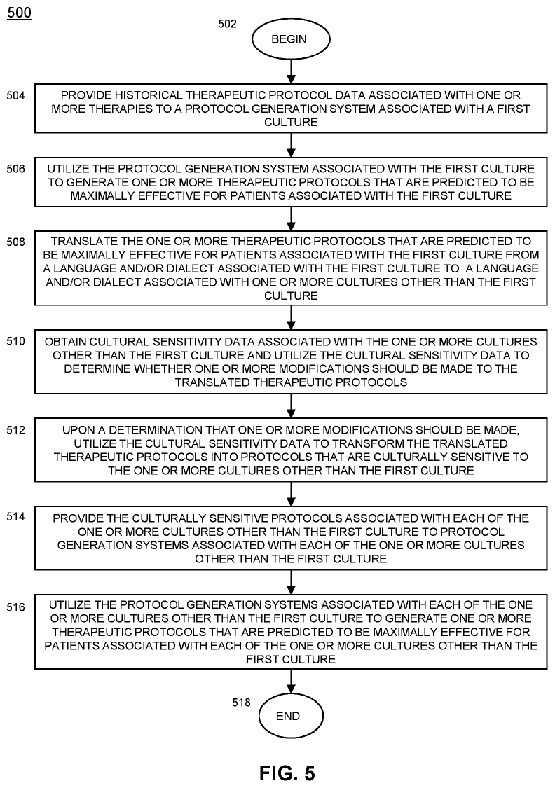

FIG. 5 is a flow chart of a process 500 for generating culturally sensitive therapeutic protocols, in accordance with one embodiment.

Process 500 begins at BEGIN 502 and process flow proceeds to 504. At 504, historical therapeutic protocol data associated with one or more therapies are provided to a protocol generation system associated with a first culture.

As noted above, and as used herein, the term “historical therapeutic protocol data” may include data associated with protocols that have previously been generated, tested, established, and/or clinically validated for use in administration of a therapy. In one embodiment, the historical therapeutic protocol data is protocol data that is associated with a therapy to be administered to a patient associated with a first culture. In various embodiments, the historical therapeutic protocol data includes data defining the historical therapeutic protocols and data indicating the historical effectiveness of the therapeutic protocols.

In one embodiment, once historical therapeutic protocols associated with one or more therapies are provided to a protocol generation system associated with a first culture at 504, process flow proceeds to 506. In one embodiment, at 506, the protocol generation system associated with the first culture is utilized to generate one or more therapeutic protocols that are predicted to be maximally effective for patients associated with the first culture.

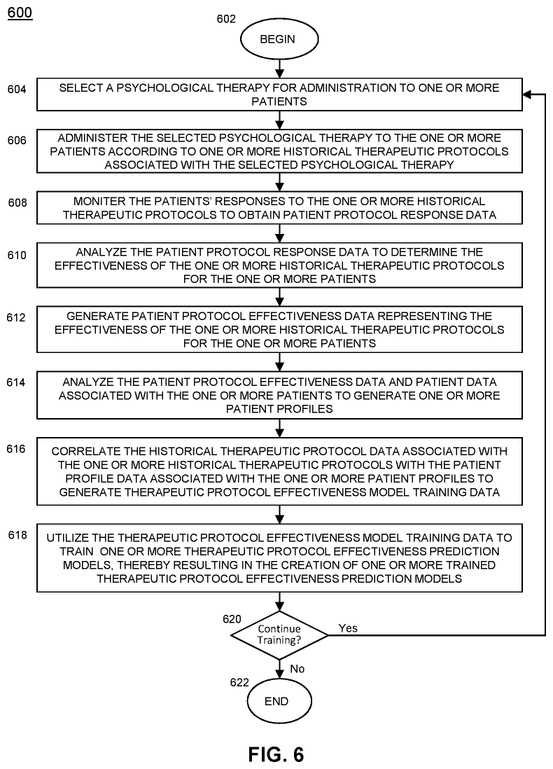

In one embodiment, the protocol generation system associated with the first culture takes historical therapeutic protocol data associated with the first culture as input data, and provides the historical therapeutic protocol data associated with the first culture to a protocol effectiveness prediction training environment, which is part of the protocol generation system associated with the first culture. The protocol effectiveness prediction training environment then generates trained therapeutic protocol effectiveness prediction models for the first culture. The process of training the therapeutic protocol effectiveness prediction models for any given culture is set forth in FIG. 6, which will be discussed in detail below.

In one embodiment, trained therapeutic protocol effectiveness prediction models associated with the first culture are then incorporated into an effective protocol generation runtime environment associated with the first culture. The effective protocol generation runtime environment associated with the first culture is then utilized to generate maximally effective therapeutic protocols for patients associated with the first culture. The process of utilizing the therapeutic protocol effectiveness prediction models to generate maximally effective therapeutic protocols for any given culture is set forth in FIG. 7 and FIG. 8, which will be discussed in detail below.

In one embodiment, once one or more maximally effective protocols for patients associated with the first culture are generated at 506, process flow proceeds to 508. In one embodiment, at 508, the one or more therapeutic protocols that are predicted to be maximally effective for patients associated with the first culture are translated from a language and/or dialect associated with the first culture to a language and/or dialect associated with one or more cultures other than the first culture.

In one embodiment, the maximally effective therapeutic protocols for patients associated with the first culture are provided to a protocol translation module, which translates the maximally effective therapeutic protocols from a language and/or dialect associated with the first culture, to a language and/or dialect associated with one or more cultures other than the first culture, resulting in translated protocol data.

In one embodiment, once the one or more maximally effective therapeutic protocols for patients associated with the first culture are translated at 508, process flow proceeds to 510. In one embodiment, at 510, cultural sensitivity data associated with the one or more cultures other than the first culture is obtained and the cultural sensitivity data is utilized to determine whether one or more modifications should be made to the translated therapeutic protocols.

In one embodiment translated protocol data is provided to a culturally sensitive protocol translation module, which utilizes cultural sensitivity data associated with one or more cultures to determine whether one or more modifications should be made to the translated protocol data to adjust for cultural sensitivities.

In one embodiment, once a determination is made as to whether one or more modifications should be made to the translated therapeutic protocols at 510, process flow proceeds to 512. In one embodiment, at 512, upon a determination that one or more modifications should be made, the cultural sensitivity data is utilized to transform the one or more translated therapeutic protocols into protocols that are culturally sensitive to the one or more cultures other than the first culture.

In one embodiment, once the cultural sensitivity data is utilized to transform the one or more translated therapeutic protocols into protocols that are culturally sensitive to the one or more cultures other than the first culture at 512, process flow proceeds to 514. In one embodiment, at 514, the culturally sensitive protocols associated with each of the one or more cultures other than the first culture are provided to protocol generation systems associated with each of the one or more cultures other than the first culture.

In one embodiment, once the culturally sensitive protocols associated with each of the one or more cultures other than the first culture are provided to protocol generation systems associated with each of the one or more cultures other than the first culture at 514, process flow proceeds to 516. In one embodiment, at 516, the protocol generation systems associated with each of the one or more cultures other than the first culture are utilized to generate one or more therapeutic protocols that are predicted to be maximally effective for patients associated with each of the one or more cultures other than the first culture.

Similarly to the functioning of the protocol generation system for the first culture, the protocol generation systems for cultures other than the first culture utilize culturally sensitive translated protocol data to generate maximally effective therapeutic protocols for patients associated with the one or more cultures other than the first culture. For example, in one embodiment, culturally sensitive translated protocol data for a second culture is provided as input data to a protocol generation system associated with the second culture, resulting in the generation of protocols that are predicted to be maximally effective for patients associated with the second culture. In one embodiment, culturally sensitive translated protocol data for a third culture is provided as input data to a protocol generation system associated with the third culture, resulting in the generation of protocols that are predicted to be maximally effective for patients associated with the third culture. In various embodiments, this operation can be performed for any number of cultures other than the first culture. The processes utilized by of each of the protocol generation systems are detailed below, in the discussion of FIG. 6, FIG. 7, and FIG. 8.

In one embodiment, once one or more maximally effective therapeutic protocols for patients associated with each of the one or more cultures other than the first culture are generated at 516, process flow proceeds to END 518 and the process 500 for generating culturally sensitive therapeutic protocols is exited to await new data and/or instructions.

FIG. 6 is a flow chart of a process 600 for creating trained therapeutic protocol effectiveness prediction models, in accordance with one embodiment.

Process 600 begins at BEGIN 602 and process flow proceeds to 604. At 604, a psychological therapy is selected for administration to one or more patients.

In one embodiment, the one or more patients are patients who have been diagnosed with a medical condition, and a determination is made as to whether the one or more patients will benefit from receiving one or more therapies. In one embodiment, once a determination has been made that one or more patients are likely to benefit from a particular therapy the therapy is selected for administration to the one or more patients.

In one embodiment, once a psychological therapy is selected for administration to one or more patients at 604, process flow proceeds to 606. In one embodiment, at 606, the selected psychological therapy is administered to the one or more patients according to one or more historical therapeutic protocols associated with the selected psychological therapy.

As noted above, there are a variety of established and/or clinically validated therapies that have been shown to provide benefit to patients, and administration of these clinically validated therapies are typically governed by a collection of therapeutic protocols associated with the particular therapy. For any given therapy, associated protocols can be defined and applied to the therapy as a whole, or to any individual component or sub-component of a therapy. In various embodiments, a determination may be made as to which historical therapeutic protocols to use for administration of the therapy to the one or more patients. This determination is at the discretion of the health care practitioner responsible for each patient's treatment, and the determination may be based on a wide variety of factors, such as, but not limited to, the severity of the patient's symptoms, the patient's medical history, the patient's age group, the patient's sex, and the patient's ethnicity.

In one embodiment, once the selected psychological therapy is administered to the one or more patients at 606, process flow proceeds to 608. In one embodiment, at 608, the patient responses to the historical therapeutic protocols are monitored to obtain patient protocol response data.

In various embodiments, patient protocol response data may include direct verbal or written feedback, and/or indirect feedback, such as an indication of whether a particular therapeutic protocol appears to be having an effect on the patient. The patient protocol response data may further include any other measureable data such as, but not limited to, click-stream data showing details related to patient engagement with the content of the therapy, for instance, the time that the patient spends engaging with each section of a particular therapy module. The patient protocol response data may also include data received from devices such as, but not limited to, sleep trackers, or other types of physiological sensors that may be used to measure a patient's physiological state, such as heart rate, respiratory rate, and/or blood pressure.

In one embodiment, once patient protocol response data is obtained at 608, process flow proceeds to 610. In one embodiment, at 610, the patient protocol response data is analyzed to determine the effectiveness of the one or more historical therapeutic protocols for the one or more patients.

In various embodiments, effectiveness of therapeutic protocols for the one or more patients may be defined and determined in a variety of ways based on the patient protocol response data. For example, in practice, patient protocol response data is typically collected in a structured manner using established clinical procedures to ensure the validity of the data interpretation. In various embodiments, the results of the data interpretation may sometimes be referred to as “clinically validated outcome measures,” which may typically be defined as tools that are used in clinical settings to assess the current status of a patient. With respect to the embodiments disclosed herein, analysis of clinically validated outcome measures for a patient can help to determine protocol effectiveness.

In one embodiment, once effectiveness of the one or more historical therapeutic protocols is determined for the one or more patients at 610, process flow proceeds to 612. In one embodiment, at 612, patient protocol effectiveness data is generated representing the effectiveness of the one or more therapeutic protocols for the one or more patients.

As detailed in the system discussion above, in various embodiments, at 612, effectiveness ratings are assigned to one or more protocols or combination of protocols, and the resulting protocol effectiveness data may be stored in one or more data structures for further use.

In one embodiment, once patient protocol effectiveness data is generated at 612, process flow proceeds to 614. In one embodiment, at 614, the patient protocol effectiveness data and patient data associated with the patient are analyzed to generate one or more patient profiles.

As discussed above, although one particular protocol or combination of protocols may be effective for the general patient population, or for the average patient, the same protocol may not be effective at all for a particular patient, or a particular type of patient, and as such, the effectiveness ratings of various protocols are likely to vary significantly depending on the characteristics of the particular patient. It follows then, that in order to train one or more therapeutic protocol effectiveness prediction models, model training data that accounts for differences in protocol effectiveness among different types of patients must be gathered and assembled. In various embodiments, the system and method disclosed herein builds a plurality of patient profiles based on known or obtained patient data. Patient data may include, for example, patient characteristics such as, but not limited to age, sex, ethnicity, religion, marital status, income level, geographic location, personal and family medical history, including the current medical issue that the therapy is designed to treat.

In one embodiment, patient protocol effectiveness data is correlated with specific profiles in the plurality of generated patient profiles, and the patient profile data may be stored in one or more data structures for later use. Any number of patient profiles may be generated, and the patient profiles are typically characterized by a combination of patient characteristics represented by the known or obtained patient data.

In various embodiments, the patient protocol effectiveness data represents a measure of how effective particular protocols are for patients of that particular type. In one embodiment, patient protocol effectiveness data may include a list of hundreds, thousands, or millions of protocols and combinations of protocols, each with corresponding data indicating an effectiveness rating for each protocol or combination of protocols. In some embodiments, an effectiveness rating for a protocol among a particular type of patient may be a single number representing an average of the effectiveness ratings for that protocol across all members of the group of patients defined by the patient profile type. In some embodiments an effectiveness rating for a protocol among a particular type of patient may be a range of numbers representing the effectiveness ratings for that protocol across all members of the group of patients defined by the patient profile type. In various other embodiments, a weighting system might be utilized, for instance to give higher weight to effectiveness ratings that are more common than others.

In one embodiment, once one or more patient profiles are generated at 614, process flow proceeds to 616. In one embodiment, at 616, historical therapeutic protocol data associated with the one or more historical therapeutic protocols is correlated with patient profile data associated with the one or more patient profiles to generate therapeutic protocol effectiveness model training data.

In various embodiments, once a plurality of patient profiles are generated and associated with protocol effectiveness data, patient profile data and historical therapeutic protocol data is collected and the data is correlated to prepare it for transformation into therapeutic protocol effectiveness model training data for training one or more therapeutic protocol effectiveness models.

In one embodiment, once therapeutic protocol effectiveness model training data is generated at 616, process flow proceeds to 618. In one embodiment, at 618, the therapeutic protocol effectiveness model training data is used to train one or more machine learning based therapeutic protocol effectiveness prediction models, thereby resulting in the creation of one or more trained therapeutic protocol effectiveness prediction models.

In various embodiments, and largely depending on the machine learning based models used, the patient profile data and/or the historical therapeutic protocol data is processed using various methods know in the machine learning arts to identify elements and to vectorize the patient profile data and/or the historical therapeutic protocol data. As a specific illustrative example, in a case where the machine leaning based model is a supervised model, the historical therapeutic protocol data and the patient profile data can be analyzed and processed to identify individual elements found to be indicative of protocol effectiveness among certain types of patients, or among a generalized population of patients. These individual elements are then used to create protocol effectiveness data vectors in multidimensional space, resulting in therapeutic protocol effectiveness model training data. The therapeutic protocol effectiveness model training data is then used as input data for training one or more therapeutic protocol effectiveness prediction models. The protocol effectiveness data for a patient profile that correlates with the protocol effectiveness data vector associated with that patient profile is then used as a label for the resulting vector. In various embodiments, this process is repeated for each protocol defined by the historical protocol definition data, and for each patient profile type represented by the patient profile data. The result is that multiple, often millions, of correlated pairs of protocol effectiveness data vectors and patient profiles are used to train the therapeutic protocol effectiveness prediction models. Consequently, this process results in the creation of one or more trained therapeutic protocol effectiveness prediction models.

In one embodiment, once one or more trained therapeutic protocol effectiveness prediction models are created at 618, process flow proceeds to 620. In one embodiment, at 620, a determination is made as to whether the one or more therapeutic protocol effectiveness prediction models should continue to be trained. In various embodiments, this determination may be made at the discretion of an operator or administrator of the system and method disclosed herein.

In one embodiment, upon a determination at 620 that the one or more therapeutic protocol effectiveness prediction models should continue to be trained, process flow returns to 604, and the above described operations may be repeated indefinitely.

In one embodiment, upon a determination at 620 that the one or more therapeutic protocol effectiveness prediction models should not continue to be trained, process flow proceeds to END 622 and the process 600 for creating trained therapeutic protocol effectiveness prediction models is exited to await new data and/or instructions.

FIG. 7 is a flow chart of a process 700 for utilizing trained therapeutic protocol effectiveness prediction models to generate maximally effective therapeutic protocols for a specific patient, in accordance with one embodiment.

Process 700 begins at BEGIN 702 and process flow proceeds to 704. At 704, a psychological therapy is selected for administration to a current patient.

In one embodiment, the current patient is a patient who has been diagnosed with a medical condition, and a determination is made as to whether the current patient will benefit from receiving one or more therapies. As noted above, there are a variety of established and/or clinically validated therapies that have been shown to provide benefit to patients, and administration of these clinically validated therapies are typically governed by a collection of therapeutic protocols associated with the particular therapy. In one embodiment, once a determination has been made that the current patient is likely to benefit from a particular therapy, that therapy is selected for administration to the current patient, and the previously trained therapeutic protocol effectiveness prediction models are utilized in order to generate one or more protocols that will be maximally effective for the current patient.

In one embodiment, once a psychological therapy is selected for administration to the current patient at 704, process flow proceeds to 706. In one embodiment, at 706, current patient data associated with the current patient and patient profile data associated with one or more predefined patient profiles are analyzed to select a patient profile that is the best match for the current patient.

In one embodiment, the current patient data is obtained, either directly from the current patient, from medical files associated with current patient, and/or current patient data may be retrieved from a database of previously collected patient data. In some embodiments, there may be no specific current patient, and test data may be used in place of current patient data. For example, a theoretical patient may be contemplated, and data describing characteristics of the theoretical patient may be used as test data in place of current patient data to generate maximally effective therapeutic protocols for the theoretical patient. In various embodiments, test data may be generated by one or more machine learning models that have been trained to predict effectiveness of the test data.

In various embodiments, once the current patient data has been obtained for the current patient, the current patient data is analyzed along with the patient profile data, in order to select a patient profile that most closely matches the characteristics of the current patient. In one embodiment, various characteristics of the current patient are compared to patient characteristics represented by the one or more patient profiles. As will be noted by those of skill in the art, various mechanisms and algorithms may be utilized to determine similarities between the current patient and the patient profiles represented by patient profile data. Similarity of the current patient to a particular patient profile may be determined by any number of factors, such as, but not limited to the current patient's age, sex, ethnicity, religion, marital status, income level, geographic location, personal and family medical history, including the current medical issue that the therapy is designed to treat. In one embodiment, one or more thresholds may be defined to determine how close of a match the current patient is to a particular patient profile.

In one embodiment, once a patient profile is selected at 706, process flow proceeds to 708. In one embodiment, at 708, the selected patient profile data is provided as input to one or more trained therapeutic protocol effectiveness prediction models.

In one embodiment, once the patient profile data is provided to one or more trained therapeutic protocol effectiveness prediction models at 708, process flow proceeds to 710. In one embodiment, at 710, new therapeutic protocol test data is generated representing one or more new therapeutic protocols associated with the psychological therapy.

In various embodiments, new therapeutic protocol test data is generated representing one or more new therapeutic protocols. As noted above, in various embodiments, new therapeutic protocols may also be thought of as potential therapeutic protocols or candidate therapeutic protocols, in the sense that they are protocols that are being considered for use in a therapy. In one embodiment, the new therapeutic protocol test data includes data representing any number of new therapeutic protocols.

In one embodiment, once new therapeutic protocol test data is generated at 710, process flow proceeds to 712. In one embodiment, at 712, the new therapeutic protocol test data is provided as input to the one or more trained therapeutic protocol effectiveness prediction models.

In one embodiment, once the new therapeutic protocol test data is provided as input to the one or more trained therapeutic protocol effectiveness prediction models at 712, process flow proceeds to 714. In one embodiment, at 714, the one or more trained therapeutic protocol effectiveness prediction models are utilized to generate predicted protocol effectiveness data for the new protocols represented by the new therapeutic protocol test data.

In various embodiments, the predicted therapeutic protocol effectiveness data represents the predicted effectiveness of each of the new therapeutic protocols represented by the new therapeutic protocol test data for a patient who matches the patient profile type represented by the selected patient profile data.

In one embodiment, once predicted protocol effectiveness data is generated at 714, process flow proceeds to 716. In one embodiment, at 716, the predicted protocol effectiveness data associated with the new therapeutic protocols and historical protocol effectiveness data associated with historical therapeutic protocols are analyzed to determine and select one or more effective therapeutic protocols.

In one embodiment, one or more of the new therapeutic protocols represented by the new therapeutic protocol test data are selected, wherein the new therapeutic protocols have been found to be effective. As discussed above, a determination as to what constitutes an “effective” protocol may be made by setting a threshold value for the effectiveness ratings represented by the predicted therapeutic protocol effectiveness data. In one embodiment, historical protocol effectiveness data may also be considered in determining and selecting effective protocols.

In one embodiment, once one or more effective therapeutic protocols are selected at 716, process flow proceeds to 718. In one embodiment, at 718, the one or more effective therapeutic protocols are utilized to generate one or more maximally effective therapeutic protocols.

In one embodiment, once one or more effective therapeutic protocols have been selected, effective therapeutic protocol definition data is generated, which contains data defining the one or more selected effective protocols. In one embodiment, the effective therapeutic protocol definition data is then used to generate one or more maximally effective therapeutic protocols. In various embodiments, the maximally effective therapeutic protocols may include any number and combination of maximally effective protocols, and each of these protocols or protocol combinations is defined by maximally effective protocol definition data.

In one embodiment, once one or more maximally effective therapeutic protocols are generated at 718, process flow proceeds to 720. In one embodiment, at 720, maximally effective protocol definition data associated with the one or more maximally effective therapeutic protocols is incorporated into historical protocol definition data for future use in administration of a psychological therapy.

Referring briefly to FIG. 6 and FIG. 7 together, in various embodiments, once one or more maximally effective therapeutic protocols have been generated, the maximally effective protocol definition data representing the maximally effective therapeutic protocols may be stored in a data structure for further use. For example, in one embodiment, maximally effective protocol definition data is stored as historical protocol definition data. This is advantageous because it creates a feedback loop for the machine learning process, wherein the newly generated maximally effective therapeutic protocols can be incorporated into the therapeutic protocol effectiveness model training data, which is generated at 616 of FIG. 6, and is used to train the therapeutic protocol effectiveness prediction models at 618 of FIG. 6. In this manner, the trained therapeutic protocol effectiveness prediction models may be continually updated and refined as new patient protocol response data is received from one or more patients.

Returning now to FIG. 7, in one embodiment, once the maximally effective protocol definition data is incorporated into historical protocol definition data at 720, process flow proceeds to 722. In one embodiment, at 722, the selected psychological therapy is administered to the patient according to the one or more maximally effective therapeutic protocols.

In one embodiment, once one or more maximally effective therapeutic protocols have been generated and/or stored, the one or more maximally effective therapeutic protocols may be incorporated into a therapy, which may then be administered to a patient, such as the current patient. In some embodiments, once generated, the maximally effective therapeutic protocols may be automatically incorporated into a therapy for administration to the current patient. In some embodiments, a health practitioner may review the maximally effective therapeutic protocols prior to incorporation into a therapy for administration to the current patient. In some embodiments, the one or more maximally effective therapeutic protocols may be incorporated into a therapy, but the therapy may not be administered to the current patient and/or the therapy may be administered to a patient other than the current patient. In various embodiments, the therapy may be administered to the current patient using one or more communication mechanisms. In some embodiments, communication mechanisms include a health practitioner conducting a physical in-person meeting with the current patient to verbally guide the current patient through the therapy. In other embodiments, communication mechanisms include administering the therapy to the current patient remotely, for example through a website, or through one or more software applications that can be executed from computing systems associated with the current patient.

In some embodiments, in addition to, or instead of, performing operations 720 and 722, the one or more maximally effective therapeutic protocols may be returned to the process described in FIG. 5 for further processing. For example, the one or more maximally effective therapeutic protocols may be associated with a first culture, and may be utilized to generate one or more protocols that are predicted to be maximally effective for cultures other than the first culture.

In one embodiment, once the selected psychological therapy is administered to the patient at 722, process flow proceeds to END 724 and the process 700 for utilizing trained therapeutic protocol effectiveness prediction models to generate maximally effective therapeutic protocols for a specific patient is exited to await new data and/or instructions.

FIG. 8 is a flow chart of a process 800 for utilizing trained therapeutic protocol effectiveness prediction models to generate generalized maximally effective therapeutic protocols, in accordance with one embodiment.

Process 800 begins at BEGIN 802 and process flow proceeds to 804. At 804, a psychological therapy is selected for administration to one or more patients.

As noted above, there are a variety of established and/or clinically validated therapies that have been shown to provide benefit to patients, and administration of these clinically validated therapies are typically governed by a collection of therapeutic protocols associated with the particular therapy. In one embodiment, a therapy is selected for future administration to one or more patients.

In one embodiment, once a psychological therapy is selected for administration to one or more patients at 804, process flow proceeds to 806. In one embodiment, at 806, new therapeutic protocol test data is generated representing one or more new therapeutic protocols associated with the psychological therapy.

In various embodiments, new therapeutic protocol test data is generated representing one or more new therapeutic protocols. As noted above, in various embodiments, new therapeutic protocols may also be thought of as potential therapeutic protocols or candidate therapeutic protocols, in the sense that they are protocols that are being considered for use in a therapy. In one embodiment, the new therapeutic protocol test data includes data representing any number of new therapeutic protocols.

In one embodiment, once new therapeutic protocol test data is generated at 806, process flow proceeds to 808. In one embodiment, at 808, the new therapeutic protocol test data is provided to the one or more trained therapeutic protocol effectiveness prediction models.

In one embodiment, once the new therapeutic protocol test data is provided to the one or more trained therapeutic protocol effectiveness prediction models at 808, process flow proceeds to 810. In one embodiment, at 810, the one or more trained therapeutic protocol effectiveness prediction models are utilized to generate predicted protocol effectiveness data for the new protocols represented by the new therapeutic protocol test data. In various embodiments, the predicted therapeutic protocol effectiveness data represents the predicted effectiveness of each of the new therapeutic protocols represented by the new therapeutic protocol test data.

In one embodiment, once predicted protocol effectiveness data is generated at 810, process flow proceeds to 812. In one embodiment, at 812, the predicted protocol effectiveness data associated with the new therapeutic protocols and historical protocol effectiveness data associated with historical therapeutic protocols are analyzed to determine and select one or more effective therapeutic protocols.

In one embodiment, one or more of the new therapeutic protocols represented by the new therapeutic protocol test data are selected, wherein the new therapeutic protocols have been found to be effective. As discussed above, a determination as to what constitutes an “effective” protocol may be made by setting a threshold value for the effectiveness ratings represented by the predicted therapeutic protocol effectiveness data. In one embodiment, historical protocol effectiveness data may also be considered in determining and selecting effective protocols.

In one embodiment, once one or more effective therapeutic protocols are selected at 812, process flow proceeds to 814. In one embodiment, at 814, the one or more effective therapeutic protocols are utilized to generate one or more maximally effective therapeutic protocols.

In one embodiment, once one or more effective therapeutic protocols have been selected, effective therapeutic protocol definition data is generated, which contains data defining the one or more selected effective protocols. In one embodiment, the effective therapeutic protocol definition data is then used to generate one or more maximally effective therapeutic protocols. In various embodiments, the maximally effective therapeutic protocols may include any number and combination of maximally effective protocols, and each of these protocols or protocol combinations is defined by maximally effective protocol definition data.

In one embodiment, once one or more maximally effective therapeutic protocols are generated at 814, process flow proceeds to 816. In one embodiment, at 816, maximally effective protocol definition data associated with the one or more maximally effective therapeutic protocols is incorporated into historical protocol definition data for future use in administration of a psychological therapy.

Referring briefly to FIG. 6 and FIG. 8 together, in various embodiments, once one or more maximally effective therapeutic protocols have been generated, the maximally effective protocol definition data representing the maximally effective therapeutic protocols may be stored in a data structure for further use. For example, in one embodiment, maximally effective protocol definition data is incorporated into historical protocol definition data. This is advantageous because it creates a feedback loop for the machine learning process, wherein the newly generated maximally effective therapeutic protocols can be incorporated into the therapeutic protocol effectiveness model training data, which is generated at 616 of FIG. 6, and is used to train the therapeutic protocol effectiveness prediction models at 618 of FIG. 6. In this manner, the trained therapeutic protocol effectiveness prediction models may be continually updated and refined as new patient protocol response data is received from one or more patients.

Returning now to FIG. 8, in one embodiment, once the maximally effective protocol definition data is incorporated into historical protocol definition data at 816, process flow proceeds to 818. In one embodiment, at 818, the psychological therapy is administered to the average patient according to the one or more maximally effective therapeutic protocols.

In one embodiment, once one or more maximally effective therapeutic protocols have been generated, the one or more maximally effective therapeutic protocols may be incorporated into a therapy, which may then be administered to an average patient. In some embodiments, once generated, the maximally effective therapeutic protocols may be automatically incorporated into a therapy for administration to the average patient. In some embodiments, a health practitioner may review the maximally effective therapeutic protocols prior to incorporation into the therapy. In some embodiments, the maximally effective therapeutic protocols may be stored in a data structure for further use, but might not be incorporated into a particular therapy. In some embodiments, the one or more maximally effective therapeutic protocols may be incorporated into a therapy, but the therapy may not be administered to an average patient. In various embodiments, the therapy may be administered to the average patient using one or more communication mechanisms. In some embodiments, communication mechanisms include a health practitioner conducting a physical in-person meeting with the average patient to verbally guide the average patient through the therapy. In other embodiments, communication mechanisms include administering the therapy to the average patient remotely, for example through a website, or through one or more software applications that can be executed from computing systems associated with the current patient.

In some embodiments, in addition to, or instead of, performing operations 816 and 818, the one or more maximally effective therapeutic protocols may be returned to the process described in FIG. 5 for further processing. For example, the one or more maximally effective therapeutic protocols may be associated with a first culture, and may be utilized to generate one or more protocols that are predicted to be maximally effective for cultures other than the first culture.

In one embodiment, once the selected psychological therapy is administered to the patient at 818, process flow proceeds to END 820 and the process 800 for utilizing trained therapeutic protocol effectiveness prediction models to generate generalized maximally effective therapeutic protocols is exited to await new data and/or instructions.

In one embodiment, a computing system implemented method comprises providing historical therapeutic protocol data associated with one or more therapies to a protocol generation system associated with a first culture, and utilizing the protocol generation system associated with the first culture to generate one or more therapeutic protocols that are predicted to be maximally effective for patients associated with the first culture. In one embodiment, a computing system implemented method further comprises translating the one or more therapeutic protocols that are predicted to be maximally effective for patients associated with the first culture from a dialect associated with the first culture to a dialect associated with one or more cultures other than the first culture, utilizing the cultural sensitivity data associated with one or more cultures other than the first culture to determine whether one or more modifications should be made to the translated therapeutic protocols, and upon a determination that one or more modifications should be made to the translated therapeutic protocols, utilizing the cultural sensitivity data to transform the translated therapeutic protocols into protocols that are culturally sensitive to the one or more cultures other than the first culture. In one embodiment, a computing system implemented method further comprises providing the culturally sensitive protocols associated with each of the one or more cultures other than the first culture to protocol generation systems associated with each of the one or more cultures other than the first culture, and utilizing the protocol generation systems associated with each of the one or more cultures other than the first culture to generate one or more therapeutic protocols that are predicted to be maximally effective for patients associated with each of the one or more cultures other than the first culture.

In one embodiment, the therapeutic protocols are associated with a therapy that includes components of one or more therapies selected from the group of therapies consisting of psychotherapy; cognitive behavioral therapy (CBT); acceptance commitment therapy (ACT); dialectical behavioral therapy (DBT); exposure therapy; mindfulness-based cognitive therapy (MCBT); hypnotherapy; experiential therapy; and psychodynamic therapy. In one embodiment, the therapy is used to treat patients diagnosed one or more health conditions selected from the group of health conditions consisting of irritable bowel syndrome (IBS); inflammatory bowel disease (IBD); gastroesophageal reflux disease (GERD); fibromyalgia; endometriosis; vulvodynia; cystitis; chronic itch; chronic cough; chronic migrate, encopresis; and constipation. In one embodiment, the therapy is administered remotely.

In one embodiment, utilizing a protocol generation system to generate one or more therapeutic protocols that are predicted to be maximally effective for patients associated with a culture includes one or more of training one or more therapeutic protocol effectiveness prediction models, utilizing one or more therapeutic protocol effectiveness prediction models to generate one or more maximally effective therapeutic protocols, and upon generation of one or more maximally effective therapeutic protocols, taking one or more actions.

In one embodiment, training one or more therapeutic protocol effectiveness prediction models includes selecting a psychological therapy for administration to one or more patients, administering the therapy to the one or more patients according to one or more historical therapeutic protocols associated with the selected psychological therapy, monitoring the responses of the one or more patients to the one or more historical therapeutic protocols to obtain patient protocol response data, analyzing the patient protocol response data to determine the effectiveness of the one or more historical therapeutic protocols for the one or more patients, and generating patient protocol effectiveness data representing the effectiveness of the one or more historical therapeutic protocols for the one or more patients. In one embodiment, a computing system implemented method further comprises analyzing the patient protocol effectiveness data and patient data associated with the one or more patients to generate one or more patient profiles. In one embodiment, a computing system implemented method further comprises correlating historical therapeutic protocol data associated with the one or more historical therapeutic protocols with patient profile data associated with the one or more patient profiles to generate therapeutic protocol effectiveness model training data. In one embodiment, a computing system implemented method comprises correlating historical therapeutic protocol data associated with the one or more historical therapeutic protocols with patient protocol effectiveness data associated with the responses of the one or more patients to the one or more historical therapeutic protocols to generate therapeutic protocol effectiveness model training data. In one embodiment, a computing system implemented method further comprises utilizing the therapeutic protocol effectiveness model training data to train one or more therapeutic protocol effectiveness prediction models, thereby resulting in the creation of one or more trained therapeutic protocol effectiveness prediction models. In one embodiment, the responses of the one or more patients to the one or more historical therapeutic protocols are monitored remotely.

In one embodiment, utilizing one or more trained therapeutic protocol effectiveness prediction models to generate one or more maximally effective therapeutic protocols includes selecting the psychological therapy for administration to a current patient, analyzing current patient data associated with the current patient and patient profile data associated with one or more predefined patient profiles to select a patient profile for the current patient, and providing selected patient profile data associated with the selected patient profile to the one or more trained therapeutic protocol effectiveness prediction models. In one embodiment, a computing system implemented method comprises generating new therapeutic protocol test data representing one or more new therapeutic protocols associated with the psychological therapy, and providing the new therapeutic protocol test data to the one or more trained therapeutic protocol effectiveness prediction models. In one embodiment, a computing system implemented method further comprises utilizing the one or more trained therapeutic protocol effectiveness prediction models to generate predicted protocol effectiveness data for the new therapeutic protocols represented by the new therapeutic protocol test data, analyzing the predicted protocol effectiveness data associated with the one or more new therapeutic protocols and historical protocol effectiveness data associated with the one or more historical therapeutic protocols to determine and select one or more effective therapeutic protocols, utilizing effective protocol definition data associated with the one or more effective therapeutic protocols to generate one or more maximally effective therapeutic protocols, and upon generation of one or more maximally effective therapeutic protocols, taking one or more actions. In one embodiment, generating one or more maximally effective therapeutic protocols includes replacing one or more of the historical therapeutic protocols with one or more of the effective therapeutic protocols.

In one embodiment, taking one or more actions includes one or more of storing maximally effective therapeutic protocol definition data associated with the one or more maximally effective therapeutic protocols for use in administration of a psychological therapy, administering the psychological therapy to one or more patients according to the one or more maximally effective therapeutic protocols, storing maximally effective therapeutic protocol definition data associated with the one or more maximally effective therapeutic protocols for incorporation into the therapeutic protocol effectiveness model training data, and incorporating the one or more maximally effective therapeutic protocols into the therapeutic protocol effectiveness model training data.

In one embodiment, the one or more therapeutic protocol effectiveness prediction models are machine learning based models that are one or more of supervised machine learning-based models, semi supervised machine learning-based models, unsupervised machine learning-based models, classification machine learning-based models, logistical regression machine learning-based models, neural network machine learning-based models, and deep learning machine learning-based models.

In one embodiment, a system comprises one or more processors and one or more physical memories, the one or more physical memories having stored therein data representing instructions which when processed by the one or more processors perform the above described computer implemented method/process.

The above described method and system result in generation of one or more culturally sensitive maximally effective therapeutic protocols, which may be incorporated into a therapy for administration to one or more patients, thus ensuring that the patients receive effective care, support, and treatment. Further, the machine learning processes described above employ a feedback loop, such that the one or more therapeutic effectiveness prediction models can be dynamically refined to account for newly received effectiveness data, thus continually improving the accuracy of future effectiveness predictions generated by the models. As a result of these and other disclosed features, discussed in detail above, the disclosed embodiments provide an effective and efficient technical solution to the technical problem of dynamically, efficiently, and rapidly generating culturally sensitive therapeutic protocols to ensure that patients associated with a wide range of cultures receive effective care, support, and treatment.

Consequently, the embodiments disclosed herein are not an abstract idea, and are well-suited to a wide variety of practical applications. Further, many of the embodiments disclosed herein require processing and analysis of billions of data points and combinations of data points, and thus, the technical solution disclosed herein cannot be implemented solely by mental steps or pen and paper, is not an abstract idea, and is, in fact, directed to providing technical solutions to long-standing technical problems associated with predicting the effectiveness of therapeutic protocols for patients associated with a wide range of cultures, and generating culturally sensitive protocols that will be maximally effective when incorporated into a therapy for administration to a patient.

Additionally, the disclosed method and system for dynamically, efficiently, and rapidly generating culturally sensitive therapeutic protocols using machine learning models requires a specific process comprising the aggregation and detailed analysis of large quantities of patient data, protocol data, protocol effectiveness data, language data, culture data, and cultural sensitivity data, and as such, does not encompass, embody, or preclude other forms of innovation in the area of culturally sensitive therapeutic protocol generation. Further, the disclosed embodiments of systems and methods for dynamically, efficiently, and rapidly generating culturally sensitive therapeutic protocols using machine learning models are not abstract ideas for at least several reasons.

First, dynamically, efficiently, and rapidly generating culturally sensitive therapeutic protocols using machine learning models is not an abstract idea because it is not merely an idea in and of itself. For example, the process cannot be performed mentally or using pen and paper, as it is not possible for the human mind to identify, process, and analyze the millions of possible patient characteristics, protocols, combinations of protocols, and associated protocol effectiveness data, along with cultural sensitivity data, and language data, even with pen and paper to assist the human mind and even with unlimited time.

Second, dynamically, efficiently, and rapidly generating culturally sensitive therapeutic protocols using machine learning models is not a fundamental economic practice (e.g., is not merely creating a contractual relationship, hedging, mitigating a settlement risk, etc.).

Third, dynamically, efficiently, and rapidly generating culturally sensitive therapeutic protocols using machine learning models is not merely a method of organizing human activity (e.g., managing a game of bingo). Rather, in the disclosed embodiments, the method and system for dynamically, efficiently, and rapidly generating culturally sensitive therapeutic protocols using machine learning models provides a tool that significantly improves the fields of medical and mental health care for patients associated with a wide range of cultures. Through the disclosed embodiments, health practitioners are provided with a tool to help them generate improved culturally sensitive therapeutic protocols, which ensures that patients are provided with personalized and maximally effective assistance, treatment, and care. As such, the method and system disclosed herein is not an abstract idea, and also serves to integrate the ideas disclosed herein into practical applications of those ideas.

Fourth, although mathematics may be used to implement the embodiments disclosed herein, the systems and methods disclosed and claimed herein are not abstract ideas because the disclosed systems and methods are not simply a mathematical relationship/formula.

It should be noted that the language used in the specification has been principally selected for readability, clarity, and instructional purposes, and may not have been selected to delineate or circumscribe the inventive subject matter. Accordingly, the disclosure of the present invention is intended to be illustrative, but not limiting, of the scope of the invention, which is set forth in the claims below.

The present invention has been described in particular detail with respect to specific possible embodiments. Those of skill in the art will appreciate that the invention may be practiced in other embodiments. For example, the nomenclature used for components, capitalization of component designations and terms, the attributes, data structures, or any other programming or structural aspect is not significant, mandatory, or limiting, and the mechanisms that implement the invention or its features can have various different names, formats, or protocols. Further, the system or functionality of the invention may be implemented via various combinations of software and hardware, as described, or entirely in hardware elements. Also, particular divisions of functionality between the various components described herein are merely exemplary, and not mandatory or significant. Consequently, functions performed by a single component may, in other embodiments, be performed by multiple components, and functions performed by multiple components may, in other embodiments, be performed by a single component.

In the discussion above, certain aspects of one embodiment include process steps and/or operations and/or instructions described herein for illustrative purposes in a particular order and/or grouping. However, the particular order and/or grouping shown and discussed herein are illustrative only and not limiting. Those of ordinary skill in the art will recognize that other orders and/or grouping of the process steps and/or operations and/or instructions are possible and, in some embodiments, one or more of the process steps and/or operations and/or instructions discussed above can be combined and/or deleted. In addition, portions of one or more of the process steps and/or operations and/or instructions can be re-grouped as portions of one or more other of the process steps and/or operations and/or instructions discussed herein. Consequently, the particular order and/or grouping of the process steps and/or operations and/or instructions discussed herein do not limit the scope of the invention as claimed below.

As discussed in more detail above, using the above embodiments, with little or no modification and/or input, there is considerable flexibility, adaptability, and opportunity for customization to meet the specific needs of various parties under numerous circumstances.

Some portions of the above description present the features of the present invention in terms of algorithms and symbolic representations of operations, or algorithm-like representations, of operations on information/data. These algorithmic or algorithm-like descriptions and representations are the means used by those of skill in the art to most effectively and efficiently convey the substance of their work to others of skill in the art. These operations, while described functionally or logically, are understood to be implemented by computer programs or computing systems. Furthermore, it has also proven convenient at times to refer to these arrangements of operations as steps or modules or by functional names, without loss of generality.

Unless specifically stated otherwise, as would be apparent from the above discussion, it is appreciated that throughout the above description, discussions utilizing terms such as, but not limited to, “activating”, “accessing”, “adding”, “aggregating”, “alerting”, “applying”, “analyzing”, “associating”, “calculating”, “capturing”, “categorizing”, “classifying”, “comparing”, “creating”, “defining”, “detecting”, “determining”, “distributing”, “eliminating”, “encrypting”, “extracting”, “filtering”, “forwarding”, “generating”, “identifying”, “implementing”, “informing”, “monitoring”, “obtaining”, “posting”, “processing”, “providing”, “receiving”, “requesting”, “saving”, “sending”, “storing”, “substituting”, “transferring”, “transforming”, “transmitting”, “using”, etc., refer to the action and process of a computing system or similar electronic device that manipulates and operates on data represented as physical (electronic) quantities within the computing system memories, resisters, caches or other information storage, transmission or display devices.

The present invention also relates to an apparatus or system for performing the operations described herein. This apparatus or system may be specifically constructed for the required purposes, or the apparatus or system can comprise a system selectively activated or configured/reconfigured by a computer program stored on a non-transitory computer readable medium for carrying out instructions using a processor to execute a process, as discussed or illustrated herein that can be accessed by a computing system or other device.

Those of ordinary skill in the art will readily recognize that the algorithms and operations presented herein are not inherently related to any particular computing system, computer architecture, computer or industry standard, or any other specific apparatus. Various systems may also be used with programs in accordance with the teaching herein, or it may prove more convenient/efficient to construct more specialized apparatuses to perform the required operations described herein. The required structure for a variety of these systems will be apparent to those of ordinary skill in the art, along with equivalent variations. In addition, the present invention is not described with reference to any particular programming language and it is appreciated that a variety of programming languages may be used to implement the teachings of the present invention as described herein, and any references to a specific language or languages are provided for illustrative purposes only and for enablement of the invention as contemplated by the inventors at the time of filing.

The present invention is well suited to a wide variety of computer network systems operating over numerous topologies. Within this field, the configuration and management of large networks comprise storage devices and computers that are communicatively coupled to similar or dissimilar computers and storage devices over a private network, a LAN, a WAN, a private network, or a public network, such as the Internet.

It should also be noted that the language used in the specification has been principally selected for readability, clarity and instructional purposes, and may not have been selected to delineate or circumscribe the inventive subject matter. Accordingly, the disclosure of the present invention is intended to be illustrative, but not limiting, of the scope of the invention, which is set forth in the claims below.

In addition, the operations shown in the figures, or as discussed herein, are identified using a particular nomenclature for ease of description and understanding, but other nomenclature is often used in the art to identify equivalent operations.

Therefore, numerous variations, whether explicitly provided for by the specification or implied by the specification or not, may be implemented by one of skill in the art in view of this disclosure.

What is claimed is:

1. A computing system implemented method comprising:
utilizing a protocol generation system associated with a first culture to generate one or more therapeutic protocols that are predicted to be maximally effective for patients associated with the first culture;
transforming the one or more therapeutic protocols that are predicted to be maximally effective for patients associated with the first culture into therapeutic protocols that are culturally sensitive to one or more cultures other than the first culture; and
utilizing one or more protocol generation systems associated with each of the one or more cultures other than the first culture to generate one or more therapeutic protocols that are predicted to be maximally effective for patients associated with each of the one or more cultures other than the first culture.

2. The computing system implemented method of claim 1 wherein the therapeutic protocols are associated with a therapy that includes components of one or more therapies selected from the group of therapies consisting of:
psychotherapy;
cognitive behavioral therapy (CBT);
acceptance commitment therapy (ACT);
dialectical behavioral therapy (DBT);
exposure therapy;
mindfulness-based cognitive therapy (MCBT);
hypnotherapy;
experiential therapy; and
psychodynamic therapy.

3. The computing system implemented method of claim 2 wherein the therapy is used to treat patients diagnosed with one or more health conditions selected from the group of health conditions consisting of:
irritable bowel syndrome (IBS);
inflammatory bowel disease (IBD);
gastroesophageal reflux disease (GERD);
fibromyalgia;
endometriosis;
vulvodynia;
cystitis;
chronic itch;
chronic cough;
chronic migrate,
encopresis; and
constipation.

4. The computing system implemented method of claim 2 wherein the therapy is administered remotely.

5. The computing system implemented method of claim 1, wherein utilizing a protocol generation system to generate one or more therapeutic protocols that are predicted to be maximally effective for patients associated with a culture includes one or more of:
training one or more therapeutic protocol effectiveness prediction models;
utilizing one or more therapeutic protocol effectiveness prediction models to generate one or more maximally effective therapeutic protocols; and
upon generation of one or more maximally effective therapeutic protocols, taking one or more actions.

6. The computing system implemented method of claim 5, wherein training one or more therapeutic protocol effectiveness prediction models includes:
administering a therapy to one or more patients according to one or more historical therapeutic protocols associated with the therapy;
analyzing patient protocol response data representing the responses of the one or more patients to the one or more historical therapeutic protocols to determine the effectiveness of the one or more historical therapeutic protocols for the one or more patients;
analyzing patient protocol effectiveness data representing the effectiveness of the one or more historical therapeutic protocols for the one or more patients and patient data associated with the one or more patients to generate one or more patient profiles;
correlating historical therapeutic protocol data, the patient protocol effectiveness data, and patient profile data associated with the one or more patient profiles to generate therapeutic protocol effectiveness model training data; and
utilizing the therapeutic protocol effectiveness model training data to train one or more therapeutic protocol effectiveness prediction models, thereby resulting in the creation of one or more trained therapeutic protocol effectiveness prediction models.

7. The computing system implemented method of claim 6 wherein the responses of the one or more patients to the one or more historical therapeutic protocols are monitored remotely.

8. The computing system implemented method of claim 5 wherein utilizing one or more trained therapeutic protocol effectiveness prediction models to generate one or more maximally effective therapeutic protocols includes:
analyzing current patient data associated with a current patient and patient profile data associated with one or more patient profiles to select a patient profile for the current patient;
providing selected patient profile data associated with the selected patient profile to the one or more trained therapeutic protocol effectiveness prediction models;
generating new therapeutic protocol test data representing one or more new therapeutic protocols associated with the therapy;
providing the new therapeutic protocol test data to the one or more trained therapeutic protocol effectiveness prediction models;
utilizing the one or more trained therapeutic protocol effectiveness prediction models to generate predicted protocol effectiveness data for the new therapeutic protocols represented by the new therapeutic protocol test data;
analyzing the predicted protocol effectiveness data associated with the one or more new therapeutic protocols and historical protocol effectiveness data associated with the one or more historical therapeutic protocols to determine and select one or more effective therapeutic protocols; and
utilizing effective therapeutic protocol definition data associated with the one or more effective therapeutic protocols to generate one or more maximally effective therapeutic protocols.

9. The computing system implemented method of claim 8 wherein generating one or more maximally effective therapeutic protocols includes replacing one or more of the historical therapeutic protocols with one or more of the effective therapeutic protocols.

10. The computing system implemented method of claim 5 wherein taking one or more actions includes one or more of:
storing maximally effective therapeutic protocol definition data associated with the one or more maximally effective therapeutic protocols for use in administration of a therapy;
administering the therapy to one or more patients according to the one or more maximally effective therapeutic protocols;
storing maximally effective therapeutic protocol definition data associated with the one or more maximally effective therapeutic protocols for incorporation into the therapeutic protocol effectiveness model training data; and
incorporating the one or more maximally effective therapeutic protocols into the therapeutic protocol effectiveness model training data.

11. The computing system implemented method of claim 5 wherein the one or more therapeutic protocol effectiveness prediction models are machine learning based models that are one or more of:
supervised machine learning-based models;
semi supervised machine learning-based models;
unsupervised machine learning-based models;
classification machine learning-based models;
logistical regression machine learning-based models;
neural network machine learning-based models; and
deep learning machine learning-based models.

12. A system comprising:
one or more processors; and
one or more physical memories, the one or more physical memories having stored therein data representing instructions which when processed by the one or more processors perform a process, the process comprising:
utilizing a protocol generation system associated with a first culture to generate one or more therapeutic protocols that are predicted to be maximally effective for patients associated with the first culture;
transforming the one or more therapeutic protocols that are predicted to be maximally effective for patients associated with the first culture into therapeutic protocols that are culturally sensitive to one or more cultures other than the first culture; and
utilizing one or more protocol generation systems associated with each of the one or more cultures other than the first culture to generate one or more therapeutic protocols that are predicted to be maximally effective for patients associated with each of the one or more cultures other than the first culture.

13. The system of claim 12 wherein the therapeutic protocols are associated with a therapy that includes components of one or more therapies selected from the group of therapies consisting of:
psychotherapy;
cognitive behavioral therapy (CBT);
acceptance commitment therapy (ACT);
dialectical behavioral therapy (DBT);
exposure therapy;
mindfulness-based cognitive therapy (MCBT);
hypnotherapy;
experiential therapy; and
psychodynamic therapy.

14. The system of claim 13 wherein the therapy is used to treat patients diagnosed with one or more health conditions selected from the group of health conditions consisting of:
irritable bowel syndrome (IBS);
inflammatory bowel disease (IBD);
gastroesophageal reflux disease (GERD);
fibromyalgia;
endometriosis;
vulvodynia;
cystitis;
chronic itch;
chronic cough;
chronic migrate,
encopresis; and
constipation.

15. The system of claim 13 wherein the therapy is administered remotely.

16. The system of claim 12, wherein utilizing a protocol generation system to generate one or more therapeutic protocols that are predicted to be maximally effective for patients associated with a culture includes one or more of:
training one or more therapeutic protocol effectiveness prediction models;
utilizing one or more therapeutic protocol effectiveness prediction models to generate one or more maximally effective therapeutic protocols; and
upon generation of one or more maximally effective therapeutic protocols, taking one or more actions.

17. The system of claim 16, wherein training one or more therapeutic protocol effectiveness prediction models includes:
administering a therapy to one or more patients according to one or more historical therapeutic protocols associated with the therapy;
analyzing patient protocol response data representing the responses of the one or more patients to the one or more historical therapeutic protocols to determine the effectiveness of the one or more historical therapeutic protocols for the one or more patients;
analyzing patient protocol effectiveness data representing the effectiveness of the one or more historical therapeutic protocols for the one or more patients and patient data associated with the one or more patients to generate one or more patient profiles;
correlating historical therapeutic protocol data, the patient protocol effectiveness data, and patient profile data associated with the one or more patient profiles to generate therapeutic protocol effectiveness model training data; and
utilizing the therapeutic protocol effectiveness model training data to train one or more therapeutic protocol effectiveness prediction models, thereby resulting in the creation of one or more trained therapeutic protocol effectiveness prediction models.

18. The system of claim 17 wherein the responses of the one or more patients to the one or more historical therapeutic protocols are monitored remotely.

19. The system of claim 16 wherein utilizing one or more trained therapeutic protocol effectiveness prediction models to generate one or more maximally effective therapeutic protocols includes:
analyzing current patient data associated with a current patient and patient profile data associated with one or more patient profiles to select a patient profile for the current patient;
providing selected patient profile data associated with the selected patient profile to the one or more trained therapeutic protocol effectiveness prediction models;
generating new therapeutic protocol test data representing one or more new therapeutic protocols associated with the therapy;
providing the new therapeutic protocol test data to the one or more trained therapeutic protocol effectiveness prediction models;
utilizing the one or more trained therapeutic protocol effectiveness prediction models to generate predicted protocol effectiveness data for the new therapeutic protocols represented by the new therapeutic protocol test data;
analyzing the predicted protocol effectiveness data associated with the one or more new therapeutic protocols and historical protocol effectiveness data associated with the one or more historical therapeutic protocols to determine and select one or more effective therapeutic protocols; and
utilizing effective therapeutic protocol definition data associated with the one or more effective therapeutic protocols to generate one or more maximally effective therapeutic protocols.

20. The system of claim 19 wherein generating one or more maximally effective therapeutic protocols includes replacing one or more of the historical therapeutic protocols with one or more of the effective therapeutic protocols.

21. The system of claim 16 wherein taking one or more actions includes one or more of:
storing maximally effective therapeutic protocol definition data associated with the one or more maximally effective therapeutic protocols for use in administration of a therapy;
administering the therapy to one or more patients according to the one or more maximally effective therapeutic protocols;
storing maximally effective therapeutic protocol definition data associated with the one or more maximally effective therapeutic protocols for incorporation into the therapeutic protocol effectiveness model training data; and
incorporating the one or more maximally effective therapeutic protocols into the therapeutic protocol effectiveness model training data.

22. The system of claim 16 wherein the one or more therapeutic protocol effectiveness prediction models are machine learning based models that are one or more of:
supervised machine learning-based models;
semi supervised machine learning-based models;
unsupervised machine learning-based models;
classification machine learning-based models;
logistical regression machine learning-based models;
neural network machine learning-based models; and
deep learning machine learning-based models.

23. A computing system implemented method comprising:
providing historical therapeutic protocol data associated with one or more therapies to a protocol generation system associated with a first culture;
utilizing the protocol generation system associated with the first culture to generate one or more therapeutic protocols that are predicted to be maximally effective for patients associated with the first culture;
translating the one or more therapeutic protocols that are predicted to be maximally effective for patients associated with the first culture from a dialect associated with the first culture to a dialect associated with one or more cultures other than the first culture;
utilizing the cultural sensitivity data associated with one or more cultures other than the first culture to determine whether one or more modifications should be made to the translated therapeutic protocols;
upon a determination that one or more modifications should be made to the translated therapeutic protocols, utilizing the cultural sensitivity data to transform the translated therapeutic protocols into protocols that are culturally sensitive to the one or more cultures other than the first culture;
providing the culturally sensitive protocols associated with each of the one or more cultures other than the first culture to protocol generation systems associated with each of the one or more cultures other than the first culture; and
utilizing the protocol generation systems associated with each of the one or more cultures other than the first culture to generate one or more therapeutic protocols that are predicted to be maximally effective for patients associated with each of the one or more cultures other than the first culture.

24. The method of claim 23 wherein utilizing a protocol generation system to generate one or more therapeutic protocols that are predicted to be maximally effective for patients associated with a culture includes:
selecting a psychological therapy for administration to one or more patients;
administering the therapy to the one or more patients according to one or more historical therapeutic protocols associated with the selected psychological therapy;
monitoring the responses of the one or more patients to the one or more historical therapeutic protocols to obtain patient protocol response data;
analyzing the patient protocol response data to determine the effectiveness of the one or more historical therapeutic protocols for the one or more patients;
generating patient protocol effectiveness data representing the effectiveness of the one or more historical therapeutic protocols for the one or more patients;
analyzing the patient protocol effectiveness data and patient data associated with the one or more patients to generate one or more patient profiles;
correlating historical therapeutic protocol data associated with the one or more historical therapeutic protocols with patient profile data associated with the one or more patient profiles to generate therapeutic protocol effectiveness model training data;
utilizing the therapeutic protocol effectiveness model training data to train one or more therapeutic protocol effectiveness prediction models, thereby resulting in the creation of one or more trained therapeutic protocol effectiveness prediction models;
selecting the psychological therapy for administration to a current patient;
analyzing current patient data associated with the current patient and patient profile data associated with one or more predefined patient profiles to select a patient profile for the current patient;
providing selected patient profile data associated with the selected patient profile to the one or more trained therapeutic protocol effectiveness prediction models;
generating new therapeutic protocol test data representing one or more new therapeutic protocols associated with the psychological therapy;
providing the new therapeutic protocol test data to the one or more trained therapeutic protocol effectiveness prediction models;
utilizing the one or more trained therapeutic protocol effectiveness prediction models to generate predicted protocol effectiveness data for the new therapeutic protocols represented by the new therapeutic protocol test data;
analyzing the predicted protocol effectiveness data associated with the one or more new therapeutic protocols and historical protocol effectiveness data associated with the one or more historical therapeutic protocols to determine and select one or more effective therapeutic protocols;
utilizing effective protocol definition data associated with the one or more effective therapeutic protocols to generate one or more maximally effective therapeutic protocols;
upon generation of one or more maximally effective therapeutic protocols, taking one or more actions.

25. The method of claim 24 wherein taking one or more actions includes one or more of:
storing maximally effective therapeutic protocol definition data associated with the one or more maximally effective therapeutic protocols for use in administration of a psychological therapy;
administering the psychological therapy to one or more patients according to the one or more maximally effective therapeutic protocols;
storing maximally effective therapeutic protocol definition data associated with the one or more maximally effective therapeutic protocols for incorporation into the therapeutic protocol effectiveness model training data; and
incorporating the one or more maximally effective therapeutic protocols into the therapeutic protocol effectiveness model training data.
